(19) United States
(12) Patent Application Publication
(10) Pub. No.: US 2009/0208955 A1
(43) Pub. Date: Aug. 20, 2009

(54) METHODS FOR IDENTIFYING SEQUENCE MOTIFS, AND APPLICATIONS THEREOF

(75) Inventors: Harlan Robins, Princeton, NJ (US); Michael Krasnitz, Princeton, NJ (US); Arnold Levine, Princeton, NJ (US)

Correspondence Address: WILMERHALE/NEW YORK, 399 PARK AVENUE, NEW YORK, NY, 10022, US

(73) Assignee: INSTITUTE FOR ADVANCE STUDY, Princeton, NJ (US)

(21) Appl. No.: 12/302,199

(22) PCT Filed: Nov. 30, 2006
(86) PCT No.: PCT/US2006/045848

§ 371 Date: Apr. 24, 2009

Related U.S. Application Data
(60) Provisional application No. 60/808,420, filed on May 25, 2006.
(60) Provisional application No. 60/830,498, filed on Jul. 13, 2006.

(30) Foreign Application Priority Data
May 30, 2006 (JP) 2006-149797

(51) Int. Cl. C12Q 1/68

(52) U.S. Cl. 435/6

(57) ABSTRACT

The present invention relates to methods and algorithms that can be used to identify sequence motifs that are either under- or over-represented in a given nucleotide sequence as compared to the frequency of those sequences that would be expected to occur by chance, or that are either under- or over-represented as compared to the frequency of those sequences that occur in other nucleotide sequences, and to methods of scoring sequences based on the occurrence of these sequence motifs. Such sequence motifs may be biologically significant, for example they may constitute transcription factor binding sites, mRNA stability/instability signals, epigenetic signals, and the like. The methods of the invention can also be used, inter alia, to classify sequences or organisms in terms of their phylogenetic relationships, or to identify the likely host of a pathogenic organism. The methods of the present invention can also be used to optimize expression of proteins.

The present application claims priority to U.S. provisional patent application Ser. No. 60/808,420, filed on May 25, 2006, Japanese patent application serial number 2006-149797, filed on May 30, 2006, and U.S. provisional patent application Ser. No. 60/830,498, filed on Jul. 13, 2006. All publications, patent applications, patents, and other references mentioned herein are incorporated by reference in their entirety.

### FIELD OF THE INVENTION

The present invention provides algorithms and methods useful for identifying “sequence motifs” that are over-represented or under-represented in a given nucleotide sequence as compared to the frequency of those motifs that would be expected to occur by chance, or to the frequency of those motifs that occurs in other nucleotide sequences. The present invention also provides, inter alia, methods of scoring and/or comparing sequences based on the occurrence of such sequence motifs, methods for classifying organisms, viruses, and nucleotide sequences based on the occurrence of such sequence motifs, methods for identifying the likely hosts of pathogenic agents based on the occurrence of such sequence motifs, and methods for optimizing nucleotide sequences for particular uses by adding, disrupting, or removing such sequence motifs.

### BACKGROUND OF THE INVENTION

Nucleotide sequences contain a wealth of information in addition to the information needed to encode proteins. For example, genomic nucleotide sequences contain transcription factor binding sites, restriction enzyme binding sites, splicing signals, mRNA stability signals, and the like. It is likely that, hidden within the nucleotide sequences of organisms, are many previously unknown but biologically significant signal sequences. The ability to identify such hidden signal sequences has been confounded by the various constraints on nucleotide sequences. Such constraints include the need to encode specific proteins, codon usage preferences, and selective pressure for particular AT/GC content. In order to identify previously hidden sequence motifs, these constraints must be factored out. The present invention addresses this need in the art by providing methods and algorithms that factor out some of these constraints, and that facilitate the identification of previously hidden “sequence motifs.”

### SUMMARY OF THE INVENTION

The present invention provides methods for identifying sequence motifs that are over-represented or under-represented in a nucleotide sequence of interest (referred to as a “real genome”) as compared to the frequency of those sequence motifs that would be expected to occur by chance, or as compared to the frequency of those sequence motifs in other nucleotide sequences. The present invention also provides, inter alia, methods of scoring and/or comparing sequences based on the occurrence of such sequence motifs, methods for classifying organisms, viruses, and nucleotide sequences based on the occurrence of such sequence motifs, methods for identifying the likely hosts of pathogenic agents based on the occurrence of such sequence motifs, and methods for optimizing nucleotide sequences for particular uses by adding, disrupting, or removing such sequence motifs.

In one embodiment, the present invention provides methods and algorithms for identifying sequence motifs.

The present invention provides a method for a identifying sequence motif by selecting a real genome sequence, generating a background genome that encodes the same amino acids, and has the same codon usage as the real genome, but is otherwise random, identifying, and counting the number of occurrences of, strings of nucleotides (or “words”) of a given length in the background genome, counting the number of occurrences of each of those words in the real genome, identifying the word most significantly contributing to the difference between the real genome and the background genome, and resealing the background genome to factor out the difference between the real genome and the background genome that was due to that word. The steps of identifying the word most significantly contributing to the difference between the real genome and the background genome, and resealing the background genome to factor out the difference between the real genome and the background genome that was due to that word, can be repeated multiple times to identify additional words contributing to the difference between the real genome and the background genome. Each time these steps are repeated, an additional word is identified. The words identified are over-represented or under-represented in the real genome as compared to the frequency of those sequences that would be expected to occur by chance, and are referred to as “sequence motifs.”

Various modifications of the above method are possible. For example, in one embodiment, the number of occurrences or the “count” for each word may be converted to a measure of the probability of occurrence of that word, and the words contributing to the difference between the probability distributions of the real and background genomes can be identified. In another embodiment multiple background genomes may be generated, and the average number of occurrences of each word may be calculated across each of the background genomes generated. In another embodiment, both of these variations may be used, such that word counts are converted to probabilities and also multiple background genomes are generated. These and other modifications of the above methods can be used in various combinations. Variations of the above methods are described in the present application, or will be apparent to those of skill in the art. All such variations are within the scope of the present invention.

The types of nucleotide sequences or “genomes” for which the above methods can be used include, but are not limited to, the genomes of eukaryotic organisms, the genomes of prokaryotic organisms, the genomes of viruses, expression vectors, plasmids, cloned cDNAs, expressed sequence tags (ESTs), and portions of such sequences.

The types of sequence motifs that can be identified using these methods include, but are not limited to, mRNA stability signals, mRNA instability signals, signals that increase the rate of transcription, signals that decrease the rate of transcription, signals involved in protein translation, protein binding sites, transcription factor binding sites, promoter sequences, enhancer sequences, repressor sequences, silencer sequences, splice sites, restriction enzyme sites, and viral latency signals.

The sequence motifs that can be identified using the methods of the invention may be useful as phylogenetic markers, because the sequence motifs are likely to occur at similar frequencies in the genomes of phylogenetically-related species.

The sequence motifs that can be identified using the methods of the invention may also be found at similar frequencies in the genomes of pathogenic agents and their hosts, and thus may be useful for determining the likely host of a pathogenic agent and/or for determining whether a host is likely to be susceptible to infection by a particular pathogenic agent.

In another embodiment, the present invention is directed to methods for optimizing the production of proteins in hosts. Such methods can be used, inter alia, to optimize the production of therapeutically useful proteins, or to optimize vaccines that contain protein-coding nucleic acid sequences so as to improve the production of the proteins in a vaccinated host.

For example, in one embodiment the present invention provides a method for optimizing the production of a protein in a host by mutating a nucleotide sequence that encodes the protein to add or create one or more sequence motifs that are over-represented in the host's genome, or to remove or disrupt one or more sequence motifs that are under-represented in the host's genome, or both, wherein the mutations result in improved production of the protein in the host.

In another embodiment, the present invention provides a method for optimizing the production of a protein in a host by identifying one or more sequence motifs that are either under-represented or over-represented in the host's genome as compared to the frequency of those sequences that would be expected to occur by chance, obtaining a nucleotide sequence encoding the protein to be expressed in the host, and mutating the nucleotide sequence to reduce the number of those sequence motifs that are under-represented in the host genome, or to increase the number of those sequence motifs that are over-represented in the host genome, or both, wherein the mutations result in improved production of the protein in the host.

In another embodiment, the present invention provides a method for optimizing the production of a protein in a host by obtaining the nucleotide sequence of at least a portion of the host genome, generating a background genome that encodes the same amino acids, and has the same codon usage as the host genome, but is otherwise random, identifying, and counting the number of occurrences of each word of a given length in the background genome, counting the number of occurrences of, each word in the host genome, identifying the word most significantly contributing to the difference between the host genome and the background genome, resealing the background genome to factor out the difference between the host genome and the background genome that was due to that word, and optionally repeating the previous two steps to identify additional words contributing to the difference between the host genome and the background genome, and then obtaining a nucleotide sequence encoding a protein to be expressed in the host and mutating the nucleotide sequence encoding the protein to either remove or disrupt one or more of sequence motifs that are under-represented in the host, or to add or create one or more sequence motifs that are over-represented in the host, or both, wherein the mutations result in improved production of the protein in the host.

The protein optimization methods of the invention can be used to optimize the expression of any protein. In some preferred embodiments, the protein whose expression is optimized is a therapeutic protein. In other preferred embodiments, the protein whose expression is optimized is an immunogenic protein, such as an immunogenic protein that can be administered to a subject as a component of a proteinaceous vaccine. In other preferred embodiments, the immunogenic protein is one that is expressed in a subject from a nucleic acid present in a vaccine composition. Examples of vaccine compositions that contain nucleic acids include, but are not limited to, attenuated viral vaccines and various vector-based vaccines.

The methods of the invention can be used to optimize the production of proteins in various hosts, including but not limited to, eukaryotes, prokaryotes, bacteria and yeasts. For example, the host may be any wild-type, mutant, or transgenic animal or plant, or any cell or cell-line derived therefrom. In certain preferred embodiments, the host is a mammal, such as a human, or a cell or cell line derived from a mammal. In other preferred embodiments, the host may be an insect cell or an insect cell line. In other preferred embodiments the host is a cellular system or culture that can be used to produce large quantities or proteins for therapeutic uses. In other preferred embodiments, the host may be a subject in need of vaccination.

In another embodiment, the present invention provides various methods for comparing and/or scoring nucleotide sequences based on the occurrence of sequence motifs.

In one embodiment, the present invention provides a method for comparing a first sequence, S1, to second sequence, S2, by identifying one or more words that are either under-represented or over-represented in the first sequence, S1, as compared to the frequency of those words that would be expected to occur by chance, determining whether any of those words are either under-represented or over-represented in the second sequence, S2, and generating a score for the similarity between S1 and S2 based on the number of words for which both S1 and S2 have the same directional bias, i.e. the number of words that are either over-represented in both S1 and S2, or are under-represented in both S1 and S2.

In another embodiment, the present invention provides a method for comparing a first sequence S1 of length s1, to a second sequence S2 of length s2, by generating a list of words that are either under-represented of over-represented in S1 as compared to the frequency of those words that occur in a background genome, B_{S}1, that encodes the same amino acids, and has the same codon usage as S1, but is otherwise random, generating a list L of words W whose under- or over-representation would be statistically significant for a coding sequence of length s2 (typically a shorter coding sequence than S1), generating a background sequence B_{S2} that encodes the same amino acids, and has the same codon usage as the sequence S2, but is otherwise random, taking a word W from the list L, adding a numerical score for that word only if the word is over-represented in both S1 and S2 compared to their respective backgrounds B_{S}1 and B_{S2}, or if the word is under-represented in both S1 and S2 compared to their respective backgrounds B_{S}1 and B_{S2}, rescaling the background B_{S2}. to factor out the effects of the word W, repeating the process for each word Win the list L, determining the number of words having a score of greater than zero, out of the total number of words in the list W, and generating a final score for the similarity between S1 and S2 based on the number of words having a score of greater than zero, out of the total number of words in the list W, wherein the higher the final score the greater the similarity between sequence S1 and sequence S2

The similarity scoring methods of the invention have various uses. Nucleotide sequences that contain many of the same sequence motifs as each other, are likely to be closely related phylogenetically. Accordingly, the scoring methods of the invention can be used to classify organisms, viruses, or nucleotide sequences, and/or to determine the phylogenetic relationships between organisms, viruses, or nucleotide sequences, or to generate phylogenetic trees. Similarly, pathogenic agents such as viruses often have many of the same genetic features as their host species. Thus, the scoring methods of the invention can also be used to determine the likely host of a pathogenic agent and/or to determine whether a host is likely to be susceptible to infection by a particular pathogenic agent.

These and other embodiments of the invention are described further in the accompanying specification, drawings, and claims.

### BRIEF DESCRIPTION OF THE DRAWINGS

FIG. 1 is a schematic illustration of a method for identifying sequence motifs according to the present invention.

FIG. 2 is a schematic illustration of an iterative word search algorithm according to the present invention.

FIG. 3 provides a bacterial phylogenetic tree for 164 bacterial species. The phylogenetic tree was generated using the methods and algorithms of the present invention. The rectangle in part (a) encloses the enterobacterial clade. Part (b) provides an expanded view of the enterobacterial clade of the tree. Results for Acinetobacter strain ADP1, Nitrosomonas europaea, Erwinia carotovora, E. coli, Salmonella enterica, Salmonella enterica serovar Typhi. Shigella flexneri, Photorhabdus luminescens, Yersinia pestis, Yersinia pseudotuberculosis, Idiomarina loihiensus, Shigella oneidensis, Vibrio cholerae, Vibrio parahaemolyyticus, and Vibrio vulnificus are shown.

### DETAILED DESCRIPTION OF THE INVENTION

### Definitions

The singular forms “a,” “an,” and “the” include plural references unless the content clearly dictates otherwise. Thus, for example, reference to a “virus” includes a plurality of such viruses.

The term “sequence motif” is used herein to refer to an oligonucleotide sequence that is over- or under-represented in a “real genome” as compared to frequency of that oligonucleotide sequence that would be expected to occur by chance, or the frequency of that oligonucleotide sequence that occurs in a “background genome.” The term “word” may be used interchangeably with the term “sequence motif.” In addition, the term “word” is used to refer to any oligonucleotide sequence regardless of whether that sequence is over-represented, under-represented, or occurs at the expected frequency. A “word” may be any string of two or more nucleotides in a nucleotide sequence. For example, certain embodiments of the invention involve identifying, and counting the number of occurrences of, every word of a certain length, such as words of 2 to 7 nucleotides, in a randomized background genome, before applying further calculations to determine which of the words are over-represented or under-represented. The over- or under-represented words are referred to as “sequence motifs.”

The term “background genome” as used herein, refers to a nucleotide sequence that shares the nucleotide constraints as a “real genome,” in terms of coding for the same amino acids as the “real genome” and having the same codon usage as the “real genome,” but that is otherwise random.

The term “real genome” as used herein, refers to any nucleotide sequence for which it is desired to identify over- and/or under-represented sequence motifs. For example, the term “real genome” encompasses both protein-coding and non-coding nucleotide sequences (typically DNA or, for some viruses, RNA) that form the genome of an organism. The term “organism” is defined, for the purposes of this invention, as including viruses. The term “real genome” as used herein encompasses both nuclear nucleic acid sequences (the “nuclear genome”) and also nucleic acid sequences located in non-nuclear organelles, such as mitochondria (the “mitochondrial genome”) or chloroplasts (the “chloroplast genome”). The term “real genome” is also used herein to refer to other nucleotide sequences for which it may be desired to identify over- and or under-represented sequence motifs, including but not limited to the nucleotide sequence of cloned cDNAs, vectors (such as expression vectors), plasmids, and any other nucleotide sequence whether naturally occurring, synthetic, mutated or otherwise manipulated. Unless stated otherwise, the term “real genome” as used herein encompasses both whole/complete genomes and “genome portions” such as individual genes within genomes or any other nucleic acid sequences that form less than the entire genomic content of an organism.

The term “organism”, as used herein, includes all multicellular and unicellular life forms such as for example, animals or animal cells, plants or plant cells, bacteria, fungi, yeasts, protozoans, protists and the like. The term “organism” also includes any living structure that contains nucleic acid and is capable of reproduction. Unless stated otherwise, the term “organism” as used herein should also be construed to encompass viruses.

The term “mutant”, as used herein, refers to a modified nucleic acid or protein that has been altered (or “mutated”) by insertion, deletion and/or substitution of one or more nucleotides or amino acids. For example, the term mutant is used to refer to nucleic acid altered to disrupt a “sequence motif”, for example by substituting one or more nucleotides in the sequence motif with another nucleotide, or inserting one or more nucleotides to disrupt the sequence motif, or deleting one or more nucleotides in the sequence motif without substituting them for other nucleotides. The term “mutating” refers to the process of making such mutants.

The term “wild type” or “WT”, as used herein, refers to nucleic acids, and to organisms, cells, viruses, vectors, and the like, that have not been manipulated artificially to disrupt a sequence motif. The term “wild type” also refers to proteins encoded by such nucleic acids. Thus, the term “wild type” includes naturally occurring nucleic acids, viruses, vectors, cells and proteins. However, in addition, the term “wild type” includes non-naturally occurring nucleic acids, viruses, cells and proteins. For example, unless otherwise stated, nucleic acids, viruses, vectors and cells that have been altered genetically are encompassed by the term “wild type” provided that those nucleic acids, viruses and cells have not been genetically altered with the intention of disrupting a sequence motif therein.

The terms “protein” and “peptide”, as used herein, refer to polymeric chain(s) of amino acids. Although the term “peptide” is generally used to refer to relatively short polymeric chains of amino acids, and the term “protein” is used to refer to longer polymeric chain of amino acids, there is some overlap in terms of molecules that can be considered proteins and those that can considered peptides. Thus, the terms “protein” and “peptide” may be used interchangeably herein, and when such terms are used they are not intended to limit in anyway the length of the polymeric chain of amino acids referred to. Unless otherwise stated, the terms “protein” and “peptide” should be construed as encompassing all fragments, derivatives, variants, homologues, and mimetics of the specific proteins mentioned, and may comprise naturally occurring amino acids or synthetic amino acids.

The term “host” refers to any organism or any cell (including, but not limited to animals, animal cells, plants, plant cells, bacteria and fungi) which may be (a) infected by an “infectious agent” or (b) used to grow and/or amplify a nucleic acid or a nucleic acid containing organism or agent, (c) which may be used to express any nucleic acid sequence or (d) which may require treatment or vaccination. Organisms in need of treatment or vaccination may also be referred to as “subjects”. The term “host” includes, inter alia, cells used to amplify viruses, vectors, or plasmids, and cells used to express recombinant proteins.

The terms “pathogen”, “pathogenic agent” and “infectious agent” are used interchangeably herein to encompass, inter alia, bacteria, viruses (including bacteriophages), fungi, yeast, protozoans (such as the malaria parasite), protists, and prions (such as the prions that cause transmissible spongiform encephalopathies such as Creutzfeldt-Jakob disease).

The terms “vaccine” and “immunogenic composition” are used interchangeably herein to refer to agents or compositions capable of inducing an immune response in a host. The terms “vaccine” and “immunogenic composition” encompass prophylactic/preventive vaccines and therapeutic vaccines. A prophylactic vaccine is one administered to subjects who are not infected with the pathogenic agent against which the vaccine is designed to protect. An ideal prophylactic vaccine will prevent a pathogenic agent from establishing an infection in a vaccinated subject, i.e. it will provide complete protective immunity. However, even if it does not provide complete protective immunity, a prophylactic vaccine may still confer some protection to a subject. For example, a prophylactic vaccine may decrease the symptoms, severity, and/or duration of a disease caused by a pathogenic agent. A therapeutic vaccine, is administered to reduce the impact of an infection in a subject already infected with a pathogenic agent. A therapeutic vaccine may decrease the symptoms, severity, and/or duration of a disease caused by a pathogenic agent.

The term “therapeutic protein” is used herein to refer to a protein that, when administered to a subject, is useful for the treatment, amelioration, or prevention of a disease or disorder. The term “immunogenic protein” is used herein to refer to a protein that, when administered to a subject, is capable of stimulating an immune response.

### Algorithms of the Invention

There are various constraints on the nucleotide sequence of genomes. One such constraint is selective pressure for particular amino acid sequences in the proteins encoded by the genome. Because the genetic code is degenerate, nucleotide sequences can theoretically differ from each other at the nucleotide level but still encode the same protein or peptide. In nature, however, there is often selective pressure for particular codon usage. For example, although two codons may encode the same amino acid, one codon may be used more frequently in a genome than another codon that encodes the same amino acid. The present invention provides methods and algorithms that normalize for each of these selection pressures, and then identify sequence motifs that are either over- and under-represented in genomes, or in genome portions, compared to the frequency of those motifs that would be expected to occur by chance. The present invention also provides scoring algorithms that can be used to classify a sequence, or compare or predict the relationship between sequences, based on the sequence motifs that they contain. These methods and algorithms are also described in Robins et al. (2005), Journal of Bacteriology, Vol. 187, p. 8370-74, the contents of which are hereby incorporated by reference. The sequence motifs of the invention may contain functional information and may be biologically significant. For example, the over- and/or under-represented sequences may be transcription factor binding sites, splice sites, mRNA degradation/stabilization signals, epigenetic signals, and the like. The over- and/or under-represented sequences may also be important in host-pathogen interactions. Thus, the methods and algorithms of the invention may be useful for identifying biologically important sequence motifs, which may then be altered to achieve certain goals.

### Algorithms for Identifying Sequence Motifs

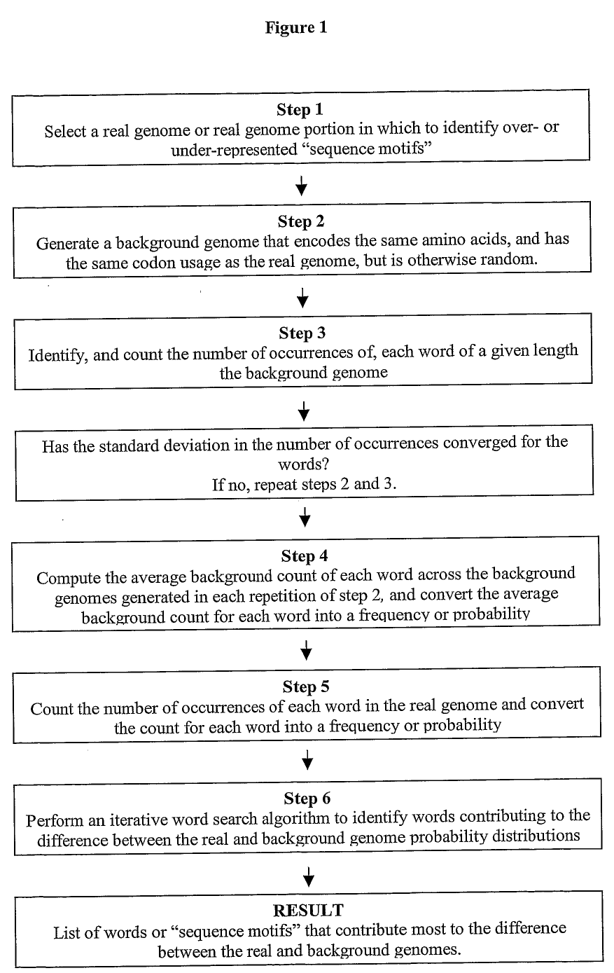

In one embodiment, the present invention is directed to a method for identifying one or more sequence motifs that are either under- or over-represented in a real genome, comprising performing the following steps. Step 1: selecting a real genome or real genome portion in which to identify under- or over-represented sequence motifs. Step 2: generating a background genome that encodes the same amino acids, and has the same codon usage as the real genome, but is otherwise random. Step 3: identifying, and counting the number of occurrences of, each word of a given length in the background genome. Steps 2 and 3 may be repeated one or more times to generate additional background genomes. Step 4: if multiple background genomes have been generated, calculating the average number of occurrences of each word across each of the background genomes generated in each repetition of step 2, and, optionally, converting the average count for each word in the background genome into a frequency or probability of that word in the background genome. Step 5: counting the number of occurrences of each of the words identified in step 3 in the real genome and, optionally, converting the count for each word in the real genome into a frequency or probability of that word in the real genome. Step 6: applying an “iterative word search algorithm” to identify one or more words contributing to the difference between the real and background genomes. The “sequence motifs” identified using this method, are “words” that are either under- or over-represented in the real genome as compared to the frequency of those words that would be expected to occur by chance. A schematic representation of this embodiment is illustrated in FIG. 1. It is preferred that the above steps are performed in the order described above. However, some of the steps may be performed in different orders, or may be performed concurrently. For example, in embodiments where steps 2 and 3 are repeated multiple times, it is not necessary to complete one iteration of steps 2 and 3 before moving on to the next iteration. Instead. Step 2 can be performed multiple times independently or simultaneously, as can step 3. Steps 4 and 5 can also be performed concurrently.

Step 1 of the above embodiment involves selecting a real genome in which to identify sequence motifs. As described in the Definitions section above, the term “real genome” is broadly defined and encompasses, inter alia, whole genomes of organisms (including viruses), portions of the whole genomes of organisms, and also any nucleotide sequence for which it is desired to identify over- and or under-represented sequence motifs, including but not limited to cloned cDNAs, vectors (such as expression vectors), plasmids, and any other nucleotide sequence whether naturally occurring, synthetic, mutated or otherwise manipulated. The nucleotide sequence of the real genome may be obtained from any source known in the art, or obtained by any suitable method known in the art. For example, the real genome sequence may be obtained from a publicly available database such as the GenBank database (available through National Center for Biotechnology Information (NCBI) at http://www.ncbi.nlm.nih.gov/), the UCSC Genome Browser (available at http://genome.ucsc.edu/cgi-bin/hgGateway) or any of the public genome project databases. The nucleotide sequence of the real genome may also be obtained from an article or publication that provides the nucleotide sequence. Alternatively, the sequence may be determined using any technique known in the art, including standard cloning and sequencing techniques. For example, if it is desired to identify over- and/or under-represented sequence motifs in a particular virus, the viral genome or portions of the viral genome, can be isolated (if necessary), cloned (if necessary) and sequenced. Suitable techniques for isolating, cloning, and determining the sequence of nucleic acids are well known in the art. See for example, Sambrook et al (2001) Molecular Cloning: A Laboratory Manual, 3rd Ed., Cold Spring Harbor Laboratory, Cold Spring Harbor, N.Y (“Sambrook”).

Step 2 of the above embodiment involves generating a background genome that encodes the same amino acids, and has the same codon usage as the real genome, but is otherwise random. The actual nucleotide molecules of the background genome need not be generated, and preferably should not be generated. Instead only virtual molecules need be generated, i.e. the sequence of the background genome should be determined, for example using a computer, but the actual nucleic acid molecule having the sequence of the background genome need not be produced. In some embodiments the real genome may consist of, or comprise, a nucleotide sequence that does not encode amino acids. For example, the real genome may consist of, or comprise nucleotide sequences that do not form part of an open reading frame (ORF), such as nucleotide sequences from regulatory regions and/or introns. In such embodiments, the background genome should ideally be random in regions corresponding to the non-coding regions of the real genome, and should encode the same amino acids and have the same codon usage as the real genome in the coding regions, but otherwise be random in the coding regions. Any suitable method for generating the background genomes of the invention can be used, for example a Monte Carlo algorithm can be used to generate permutations of the real genome sequence that still encode the same amino acids as the real genome and still employ the same codon usage, but are otherwise random. In a preferred embodiment, the Monte Carlo algorithm created by Fuglsang to resample codons in genes while keeping the amino acid sequence of the translation product constant, is used. See Fuglsang, (2004) “The relationship between palindrome avoidance and intragenic codon usage variations: a Monte Carlo study” Biochem. Biophys. Res. Commun. 316: 755-762, the contents of which are hereby incorporated by reference.

Step 3 of the above embodiment involves identifying, and counting the number of occurrences of, each word of a given length in the background genome. A word must contain at least two nucleotides, but the upper limit on word length is variable. One of skill in the art can select a suitable range of word lengths, depending on factors such as the total size of the real genome, and the computing power available. For example, consider the situation where 2 is chosen as the minimum word length and 5 is chosen as the maximum word length. The total number of words of between 2 and 5 nucleotides in a 10 nucleotide long real genome is small, and a computer can therefore easily identify and count all of the possible words. However, the total number of words of between 2 and 5 nucleotides in length in the human genome (which is approximately 3 billion base pairs long) is very large, and it would therefore require significant computing power to identify and count all of such words. The greater the size of the real genome being studied, the greater the computing power needed. Similarly, the greater the range of word lengths, the greater the computing power needed. Time is also a factor to be considered. The more computations that are needed to identify all of the words of a given length in a real genome, the longer the computation will take to perform. Another factor that should be taken into account in selecting suitable word lengths, is the number of occurrences of words of that length in the background genome. Ideally, the average number of occurrences of each word should be much greater than zero in order for the algorithms of the invention to operate in a robust manner. The longer the length of the word, the lower the occurrence of that word will be. For example, a 20-letter word will occur fewer times than a 2 letter word. The range of word lengths should be chosen such that, in the genome being analyzed, words of those lengths will occur more than 10-20 times.

One of skill in the art can readily select a suitable lower and upper limit on word length taking these considerations into account. For example, in the Examples provided herein, a minimum word length of 2 nucleotides and a maximum word length of 7 nucleotides was chosen for analysis of the entire genomes of several bacterial species. Longer word lengths could have been chosen if desired, taking into account the above considerations.

Once a suitable word length, or range of word lengths, has been chosen, routine methods can be used to identify and count each word. For example the nucleotide sequence AGCTCA contains the 2 “letter” words AG, GC, CT, TC, and CA, the 3 letter words AGC, GCT, CTC, and TCA, and the four letter words AGCT, GCTC, CTCA. Thus, there is a list of 12 words having a maximum length of 4 nucleotides, in the sequence AGCTCA when reading from the 5′ to 3′ direction (and assuming the sequence is not circular), each of these words occurring only once. This type of word identification and word counting can be performed using standard methods known in the art in order to identify and count words of a given length in a given real genome.

In a preferred embodiment, steps 2 and 3 should be repeated multiple times, i.e. more than one background genome should be generated, and the words of a given length in each background genome generated should be identified and counted. Each time step 2 is repeated, it is possible that more words will created by random permutation. The more background genomes that are generated, the more statistically robust/representative the words and word counts will be. The procedure to generate random genomes can be repeated as many times as desired. In a preferred embodiment, the procedure to generate random genomes is repeated more than 5 times, more preferably more than 5-10 times, more preferably more than 10-20, more preferably more than 20-30 times, or more preferably more than 30-40 times. However, the number of times that the procedure to generate background genomes is repeated can be selected depending on factors such as the length of words to be identified, the size of the real genome, and the like. In a preferred embodiment, the procedure to generate random genomes is repeated until the standard deviation of the number of occurrences of the words converges. At this point, the words and word counts will be statistically robust/representative.

Step 4 of the above embodiment involves calculating the average number of occurrences of each word across each of the background genomes generated in each repetition of step 2. In one embodiment, this is done by simply counting the total number of occurrences of a given word across all of the background genomes generated, and then dividing that number by the total number of background genomes to give the average background count of that word across all of the background genomes.

In another embodiment, it is possible to calculate the average word count by considering only words of a given length (such as the maximum length) and then generating the counts for smaller-length words by counting substrings. For example, for words of up to 7 nucleotides in length, the average word count can be calculated by considering only the words of 7 nucleotides in length, and then generating the counts for smaller-length words by counting substrings. Any suitable method for performing this calculation can be used. For example, in a preferred embodiment, the average background count, N_{B}(w), can be calculated as follows.

Let L(w) equal the length of the word w, and let C(W₇ⁱ, w) equal the number of times the string w is contained in the string W₇ⁱ of length 7. As an example, if w is AAC and W₇²⁵⁷ is AACAAAC, then L(w) equals 3 and C(W₇²⁵⁷, w) equals 2. This is example is based on a maximum word length of 7 nucleotides, but other word lengths can also be used, as desired.

If 30 background genomes are generated, the average background count for a given word of 7 nucleotides in length across each of the background genomes, N_{B}(W₇ⁱ), is equal to 1/30×(the sum of the number counts of that word, W₇ⁱ, in each of the 30 background genomes). The average background count for each word, N_{B}(w), was calculated according to equation (1) below.

$${N}_{B} ( w ) = {\sum }_{i = 0}^{( {4}^{7} - 1 )} \frac{{N}_{B} ( {W}_{7}^{i} ) \times C ( {W}_{7}^{i} , w )}{8 - L ( w )} \qquad ( 1 )$$

Note that, although the above description and equation refers to, or is used for, words of up to 7 nucleotides in length, the description and equations can be adapted for words of any desired length.

In a preferred embodiment, the counts for each word in the background genome are then converted to frequencies (or equivalently probabilities). For example, this can be performed using the formula P_{B}(W)=N_{B}(w)/L, where P_{B}(w) is the probability of word w being present, N_{B}(w) is the average background of the word w, and L is the overall length of the background genome.

Step 5 of the above embodiment involves counting the number of occurrences of each of the words identified in step 3, in the real genome. This can be performed by simple counting, as generally only one real genome is considered at any one time, and thus there is no need to produce average counts. This can be done using standard methods known in the art in order to identify and count words of a given length in a given real genome. As for step 4, in a preferred embodiment, the counts for each word in the real genome are then converted to frequencies (or equivalently probabilities). For example, this can be performed using the formula P_{R}(w)=N_{R}(w)/L, where P_{R}(w) is the probability of word w being present in the real genome, N_{R}(w) is the count of word w in the real genome, and L is the overall length of the real genome.

Step 6 of the above embodiment involves applying an “iterative word search algorithm” to identify words contributing to the difference between the real and background genome probability distributions. The words or “sequence motifs” identified using this method are words that are either under- or over-represented in the real genome as compared to the frequency of those words that would be expected by chance. Any suitable algorithm capable of identifying words contributing to the difference between the real and background genome probability distributions can be used.

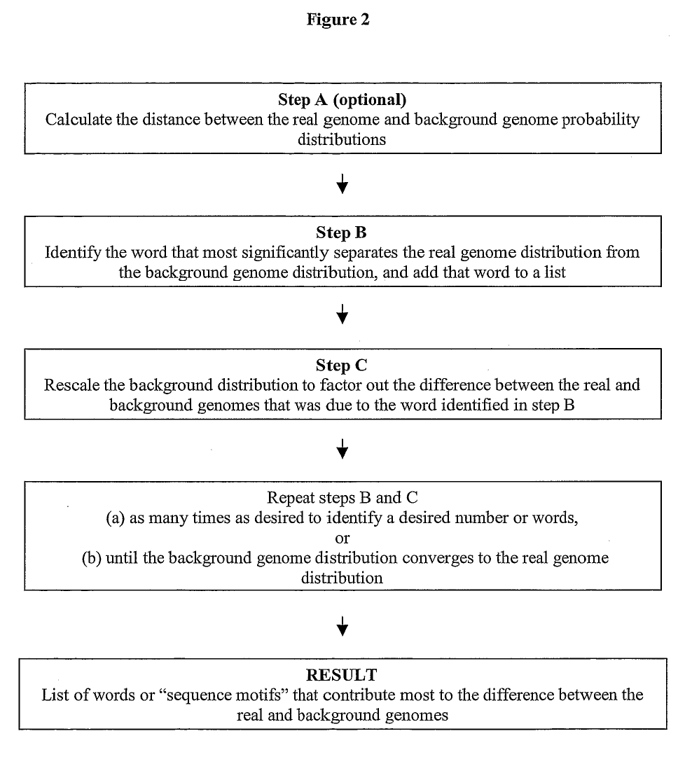

In a preferred embodiment, the “iterative word search algorithm” used is one of those described herein, which involves performing the following steps. Step A: an optional first step of calculating the distance between the real genome and background genome probability distributions. Step B: identifying the word that most significantly separates the real genome distribution from the background genome distribution. Step C: rescaling the background distribution to factor out the difference between the real and background genomes that was due to the word identified in step B. Steps B and C may be repeated either as many times as desired to identify a desired number or words, or until the background genome converges to the real genome. The words or “sequence motifs” identified using these steps are words that are either under- or over-represented in the real genome as compared to the frequency of those words that would be expected by chance. The steps of this iterative word search algorithm are illustrated in FIG. 2.

Step A of the above iterative word search algorithm involves calculating the distance between the real genome and background genome probability distributions. This step is useful for monitoring purposes (subsequent steps should decrease the distance between the real and background genomes), but is optional. Any method known in the art for calculating the distance between two probability distributions can be used. Such methods include, but are not limited to, the Kullback-Leibler method, the _{χ}2-statistic method, the quadratic form distance method, the match distance method and the Kolmogorov-Smirnov distance method. One of skill in the art can readily select and apply any such method to determine the “distance” between the real and background genome distributions.

In a preferred embodiment, the Kullback-Leibler method is used. The Kullback-Leibler distance, also known as information divergence, information gain, or relative entropy, is a natural distance measure from a “true” probability distribution P to an arbitrary probability distribution Q. Typically P represents data, observations, or a precise calculated probability distribution. The measure Q typically represents a theory, a model, a description or an approximation of P. It can be interpreted as the expected extra message-length per datum that must be communicated if a code that is optimal for a given distribution Q is used, compared to using a code based on the true distribution P. For probability distributions P and Q of a discrete variable, the K-L distance (D_{KL}) of Q from P is defined to be

$${D}_{KL} ( P ‖ Q ) = {\sum }_{i} P ( i ) log \frac{P ( i )}{Q ( i )}$$

See Kullback, S., and R. A. Leibler, 1951, “On information and sufficiency” Annals of Mathematical Statistics 22: 79-86, the contents of which are hereby incorporated by reference, for further description of the Kullback Leibler method. For the purposes of the present invention, the Kullback-Leibler distance D_{KL} between the real genome and background genome probability distributions can be calculated using equation (2) below.

$${D}_{KL} = {\sum }_{i} {P}_{R} ( {W}_{7}^{i} ) log \frac{{P}_{R} ( {W}_{7}^{i} )}{{P}_{B} ( {W}_{7}^{i} )} \qquad ( 2 )$$

Note that, although the above equation refers to words of up to 7 nucleotides in length, the same equation can be used for words of any desired length.

Step B of the above iterative word search algorithm involves identifying the word that most significantly separates the real genome distribution from the background genome distribution. This can be performed using any suitable method known in the art. In a preferred embodiment, this is performed by producing a score to measure the significance of the contribution of each word to the difference between the two distributions, or S(w). S(w) measures the extent to which any one word w, of a given length, contributes to D_{KL} (i.e. contributes to the difference between the background probability P_{B} and the real probability P_{R}). In a preferred embodiment, S(w), is calculated using equation (3) below.

$$S ( w ) = {P}_{R} ( w ) log \frac{{P}_{R} ( w )}{{P}_{B} ( w )} + [ 1 - {P}_{R} ( w ) ] log ( \frac{1 - {P}_{R} ( w )}{1 - {P}_{B} ( w )} ) \qquad ( 3 )$$

Step C of the above iterative word search algorithm involves resealing the background distribution to factor out the difference between the real and background genomes that was due to the word identified in step B. This may be done by any suitable method known in the art. It is preferred that this is done in a minimal way such that the contribution of w becomes identical in both the real and background distributions, i.e. to factor out the contribution of w to the background genome. For the resealing to be minimal, it is preferred that the ratios of frequencies of words Wⁱ_{X} of length x that contain w the same number of times should not change. That is, all words Wⁱ_{X} with the same C(Wⁱ_{X},w) are preferably rescaled by an equal factor. To accomplish this, it may be necessary to work with an appropriate coarse graining of the detailed probability distributions.

In a preferred embodiment, the distribution for the background should be defined as the set of words Wⁱ_{X} of length X, with the probabilities P_{B}(Wⁱ_{X}) and this set of Wⁱ₇ should be partitioned into disjoint subsets where each element of a given subset contains the word w an equal number of times. Equations (4) and (5) below provide preferred definitions of these subsets.

K_{J}(w)={W₇ⁱ|C(W₇ⁱ,w)=J} (4)

with J={0, . . . , 6} and

K₀∪ . . . ∪K₆={W₇ⁱ} (5)

In the above equations J is an integer that is the number of times that a short word (w) occurs in a long word (W). For example, for the 7 letter word (W⁷) “ACGGACT” and the short word (w) “AC”, the number of occurrences of w in W⁷ is 2 (i.e. C (W⁷, w)=2) and so J is 2. K is the set of all words of length 7 which contain the word (w) J times.
The disjoint subsets K_{J}(W) should be rescaled such that the probabilities of being in a given subset in the real and background distributions are equal, as illustrated by equations (6) and (7) below.

$$\begin{matrix}{Q}_{R} ( {K}_{J} ) = {\sum }_{{W}_{7}^{i} ∈ {K}_{J}} {P}_{R} ( {W}_{7}^{i} ) & ( 6 ) \\ {Q}_{B} ( {K}_{J} ) = {\sum }_{{W}_{7}^{i} ∈ {K}_{J}} {P}_{B} ( {W}_{7}^{i} ) & ( 7 )\end{matrix}$$

Q_{R} of the set K_{J} is the sum of the probabilities of occurrence of all the words in the set K_{J} in the real genome, and Q_{B} is the sum of the probabilities of occurrence of all the words in the set K_{J} in the background genome.

The above are well-defined probability distributions because they are grouped elements from the old probability distribution, and their probabilities are added. A rescaling that factors out the contribution of w while conserving probability is given by

$${N}_{B} ( {W}_{7}^{i} ) \to \frac{{Q}_{R} ( {K}_{J} )}{{Q}_{B} ( {K}_{J} )} {N}_{B} ( {W}_{7}^{i} ) \qquad ( 8 )$$

where Wⁱ₇εK_{J}, for all i. Note that with this rescaled distribution, the figure of merit for w is now zero (S^{rescaled}(w)=0) because the contribution of w to the difference between the real and background genomes has been factored out. Stated another way, the contribution of w to D_{KL} has been removed.

In a preferred embodiment, steps B and C should be repeated. The steps can be repeated either as many times as desired to identify a desired number or words, or until the background genome converges to the real genome. Thus, after identifying a first word that most significantly contributes to the difference between the real and background distributions in step B above, and then resealing the background genome to factor out the contribution of this word, step B should be repeated to find a second word, w′, which contributes most to the difference between the real and rescaled background genomes. After identifying the second word, w′, step C should then be repeated to factor out the contribution of word w′, before repeating step B to find a third word, w″, and so on.

With each successive round of this iterative algorithm, the background distribution will converge to the real distribution. This is because D_{KL} monotonically decreases for successive iterations until D_{KL} is 0 (see Example 2), which occurs only if the two distributions are identical. In one embodiment, steps B and C are be repeated until convergence between the background distribution and real distribution is achieved, i.e. until the equation S(w)=0 for all w, and D_{KL} is 0, which will occur when the real and background distributions are identical.

However, in another embodiment the algorithm may be stopped or cut-off at any desired stage or after a desired number of iterations of steps B and C. For example, in a preferred embodiment, the algorithm, is stopped at a point where the iterations are no longer contributing statistically significant words to the list. In another preferred embodiment, the algorithm is stopped at the point where it becomes likely that chance fluctuations would create the most significant remaining word(s). This cut-off point occurs when the selected word w satisfies equation (9) below, where “erfc” refers to the well-known statistical function known as the complementary error function.

$$erfc \{ \frac{[ {N}_{R} ( w ) - {N}_{B} ( w ) ]}{Δ ( w ) \sqrt{2}} \} \times {4}^{L ( w )} > 1 / 2 \qquad ( 9 )$$

In another preferred embodiment, the algorithm is stopped after any desired number of iterations or when the desired number of sequence motifs have been identified. Using the above method, each iteration identifies one sequence motif that is either over- or under-represented in the real genome. Thus, if it is desired to identify 10 sequence motifs, the algorithm can be stopped after 10 iterations, or if it is desired to identify 50 sequence motifs, the algorithm can be stopped after 50 iterations, or if it is desired to identify 100 sequence motifs, the algorithm can be stopped after 100 iterations, and so on. In the Examples provided below, the algorithms were stopped after 100 iterations, which was substantially below the cutoff for those algorithms that was calculated using equation (9).

### Scoring Algorithms

The present invention also provides methods and algorithms that can be used to score a coding sequence, S, of length s, with respect to a genome G of length g (or, stated another way, a first sequence S1 of length s1 with respect to a second sequence S2 of length s2). Such methods are useful for many applications. For example, in one embodiment, an unknown sequence can be classified in terms of the organism/species that the sequence derives from, using the scoring methods of the invention. In another method, the scoring methods can be used to determine the evolutionary relationship between different sequences or genomes and thereby create a phylogenetic tree. In another embodiment, the scoring methods can be used to identify the likely host of a pathogenic agent such as a virus, or to identify pathogenic agents that are likely to infect a certain host. These and other applications of the scoring methods and algorithms of the invention are described in more detail below.

In one embodiment, the present invention provides a method for comparing a first sequence, S1, to second sequence, S2, by identifying one or more words that are either under-represented or over-represented in the first sequence, S1, as compared to the frequency of those words that would be expected to occur by chance, determining whether any of those words are either under-represented or over-represented in the second sequence, S2, and generating a score for the similarity between S1 and S2 based on the number of words for which both S1 and S2 have the same directional bias, i.e. the number of words that are either over-represented in both S1 and S2, or are under-represented in both S1 and S2. In preferred embodiments, the words that are either under-represented or over-represented are identified using one of the sequence motif identifying algorithms described herein.

In another embodiment, the present invention provides a method for comparing a first sequence S1 of length s1, to a second sequence S2 of length s2, where S2 is longer than S1, by generating a list of words that are either under-represented of over-represented in S2 as compared to the frequency of those words that occur in a background genome, B_{S2}, that encodes the same amino acids, and has the same codon usage as S2, but is otherwise random, generating a list L of words W, whose under- or over-representation would be statistically significant for a coding sequence of length s1 (typically a shorter coding sequence than S2), generating a background sequence B_{S1} that encodes the same amino acids, and has the same codon usage as the sequence S1, but is otherwise random, taking a word W from the list L, adding a numerical score for that word only if the word is over-represented in both S1 and S2 compared to their respective backgrounds B_{S1} and B_{S2}, or if the word is under-represented in both S1 and S2 compared to their respective backgrounds B_{S1} and B_{S2}, resealing the background B_{S2} to factor out the effects of the word W, repeating the process for each word W in the list L, determining the number of words having a score of greater than zero, out of the total number of words in the list W, and generating a final score for the similarity between S1 and S2 based on the number of words having a score of greater than zero, out of the total number of words in the list W, wherein the higher the final score the greater the similarity between sequence S1 and sequence S2. As above, in a preferred embodiment, the words that are either under-represented or over-represented are identified using one of the sequence motif identifying algorithms described herein.

In another embodiment, the present invention provides a method to score a coding sequence, S, of length s, with respect to a genome G of length g (or, stated another way, a first sequence S1 of length s1 with respect to a second sequence S2 of length s2) wherein the method is based on the sequence motif identifying algorithms described above, with the modification that words are added to the word list only if they would be significant for a sequence of length s. The length of s is typically much shorter than the length of the genome G, and thus fewer words make it on to the list. This may be achieved by rescaling the counts and the standard deviations for each word to the scale s. For example, the counts for each word in the background genome and the real genome may be multiplied by s/g (or s1/s2), which gives the expected counts, N_{b} and Nᵣ, for the words in the sequence S of length s. The standard deviation can be rescaled by the factor √s/g, to give Δ^{s}. If a given word satisfies the equation |Nᵣ−N_{b}|>3×Δ^{s} then it is included on the list; otherwise, it is skipped. Because s is much less than g, this standard is substantially more strict than the general sequence motif identifying algorithms described herein. The rest of the iterative algorithm, including the rescaling the background distribution, may be performed in the same was as for the general sequence motif identifying algorithms described herein.

The list of words identified using the scoring method, L, forms the scoring template and has X number of words. To produce the score, the background B of the sequence S is generated using the same methods described above for generating background genomes. Then the following iterative algorithm is implemented: at each step, a word W from the ordered list L is taken, and the counts of that word in the sequence S and the background B are compared, adding a numerical score (e.g. a score of one) only if the direction of the bias for W between S and B is the same as that for W between the genome G and its background, that is, only if W is over-represented in both G and S compared to their respective backgrounds, or is under-represented in both. Then the background B is rescaled in the manner described for the general sequence motif identifying algorithm to factor out the effects of W, and the process is repeated through the entire list L. Going through the entire list L, produces a number of words Y out of X possible words for which there is agreement between the genome and the sequence. A final score may be calculated using the formula C×(X−Y/2)√Y, where C is a constant.

### Computer Systems

The methods and algorithms of the present invention are preferably performed using a computer. In one embodiment, the invention involves the use of a computer system which is adapted to allow input of the sequence of a “real genome” and which includes computer code for performing one or more of the steps of the various algorithms described herein. For example, the present invention encompasses a computer program that includes code for performing one or more of generating a background genome, counting the number of occurrences of each word of a given length the background genome, computing the average background count of each word across multiple background genomes, converting average background counts for a given word into a frequency/probability, counting the number of occurrences of a given word in a real genome, converting the count for a given word in a real genome into a frequency or probability, performing an iterative word search algorithm to identify a list of words contributing to the difference between the real and background genomes, calculating the distance between a real genome probability distribution and a background genome probability distribution, identifying words that significantly separate a real genome distribution from a background genome distribution, resealing a background genome distribution to factor out the difference between the real and background genomes due to a particular word, and the like.

The computer systems of the invention preferably comprise a means for inputting data such as the sequence of a real genome, a processor for performing the various calculations described herein, and a means for outputting or displaying the result of the calculations. Typically, that result will be a list of sequence motifs that are either over- or under-represented in a real genome as compared to a background genome.

One of skill in the art can readily create computer code for executing the methods and algorithms of the invention, using any suitable computer code language or system known in the art, such as “C” for example.

### Applications of the Algorithms and Methods of the Invention

The algorithms and methods of the present invention have many different uses and applications, some of which are described below. Other applications will be well known to those of skill in the art.

### Optimization of Sequences for Protein Production

Recombinant proteins have many applications, for example as therapeutic agents and as components of proteinaceous vaccines. These recombinant proteins are generally produced in host cells that have been transformed or transfected with expression vectors containing a nucleotide sequence that encodes the protein, under the control have a suitable promoter. Often recombinant proteins are expressed and produced in cell types of a species different than that from which the nucleotide sequence is derived. For example Amgen's recombinant human erythropoietin product is produced in cultured hamster ovary (CHO) cells, and recombinant human G-CSF, the active ingredient in the commercial product Neupogen®, is produced in E. Coli bacterial cells. In such situations, the nucleotide sequence encoding the recombinant protein may not contain certain sequence motifs that are present in the genome of the host cells, or may contain additional sequence motifs that are absent in the host cell. These differences may adversely affect the expression of foreign recombinant proteins in host cells. For example, the host genome may contain certain sequence motifs required for mRNA stabilization in the host that are absent in the recombinant nucleotide sequence, or the recombinant nucleotide sequence may contain certain sequence motifs that inhibit or decrease the efficiency of protein expression in the host. Thus, it may be useful to mutate the nucleotide sequence encoding the recombinant protein to add one or more of the host-specific sequence motifs or to remove one or more of the source species sequence motifs, so as to optimize production of the recombinant protein in the host cells. For example, if a recombinant human protein is to be expressed in hamster cells, it may be desirable to add one or more hamster-specific sequence motifs to the nucleotide sequence that encodes the recombinant human protein. Similarly, if a recombinant human protein is to be expressed in insect cells, such as using the baculovirus expression system, it may be desirable to add one or more insect-specific sequence motifs to the nucleotide sequence that encodes the recombinant human protein.

There are many variations on the above concepts, all of which are within the scope of the present invention. For example, any nucleotide sequence encoding a recombinant protein may be optimized using the methods described herein, including, but not limited to, sequences encoding any eukaryotic, prokaryotic, plant, animal, bacterial, yeast, insect, mammalian, primate, human, hamster, mouse, goat, sheep, bird or chicken recombinant protein.

Similarly, the host system in which the recombinant nucleotide protein is to be produced may be any suitable cellular expression system known in the art, including, but not limited to, eukaryotic expression systems, prokaryotic expression systems, plant expression systems, animal expression systems, bacterial expression systems, yeast cell expression systems, insect cell expression systems, mammalian cell expression systems, primate cell expression systems, human cell expression systems, hamster cell expression systems, mouse cell expression systems, goat cell expression systems, sheep cell expression systems, bird cell expression systems, chicken cell expression systems, and the like. The host expression system may also be any cell line suitable for recombinant protein expression, including, but not limited to, chinese hamster ovary (CHO) cells, mouse myeloma NS0 cells, baby hamster kidney cells (BHK), human embryo kidney 293 cells (HEK-293), human C6 cells, Madin-Darby canine kidney cells (MDCK) and Sf9 insect cells. The expression system may also be an entire organism, such as a transgenic plant or animal. For example, the expression system may be a transgenic sheep or cow that capable of expression of recombinant proteins that are secreted into the milk, or a recombinant plant capable of expressing recombinant proteins. Any suitable host system for recombinant protein expression known in the art can be used in accordance with the methods of the present invention.

As stated above, the nucleotide sequence encoding the recombinant protein can be altered in multiple ways to make it more compatible with the host's cellular environment. In a preferred embodiment, the methods of the present invention are used to identify sequence motifs present in the nucleotide sequence encoding the recombinant protein that are either over- or under-represented in the host genome. It is preferred, that in a next step, the functional consequences of the sequence motifs are determined. This can be done by mutating the sequence motifs in either the nucleotide sequence encoding the recombinant protein or in the host genome, and testing the effect of these mutations on certain biological properties such as rate of mRNA production, stability of mRNA, rate or protein production, protein stability, cleavage by restriction enzymes, and the like. In a further step it is preferred that the nucleotide sequence encoding the recombinant protein is then “optimized” by making mutations to remove or disrupt one or more disadvantageous sequence motifs or to add or create one or more advantageous sequence motifs.

For example, if a sequence motif is identified that is under-represented in the nucleotide sequence encoding the recombinant protein as compared to the frequency of that sequence motif in the host, and that sequence motif is found to increase the rate of mRNA production, increase stability of mRNA, increase the rate or protein production and/or increase protein stability in the host, then the nucleotide encoding the recombinant protein should be mutated to create one or more additional copies of that sequence motif. In a preferred embodiment, the mutations are made such that they do not alter the amino acid sequence of the protein encoded by the nucleotide sequence. If the mutations do alter the amino acid sequence of the protein encoded by the nucleotide sequence, it is preferred that the amino acid changes have no deleterious effect on the protein, or that the amino acid changes have a beneficial effect on the protein. Any suitable mutation methods known in the art, such as those described herein, may be used.

Conversely, if a sequence motif is identified that is over-represented in the nucleotide encoding the recombinant protein as compared to the frequency of that sequence motif in the host, and that sequence motif is found to decrease the rate of mRNA production, decrease stability of mRNA, decrease the rate or protein production and/or decrease protein stability in the host, then the nucleotide encoding the recombinant protein should be mutated to remove one or of these sequence motifs. In a preferred embodiment, the mutations are made such that they do not alter the amino acid sequence of the protein encoded by the nucleotide sequence. If the mutations do alter the amino acid sequence of the protein encoded by the nucleotide sequence, it is preferred that the amino acid changes have no deleterious effect on the protein, or that the amino acid changes have a beneficial effect on the protein. Any suitable mutation methods known in the art, such as those described herein, may be used.

### Optimization of Vector Sequences

In another embodiment, the algorithms and methods of the invention can be used to optimize the sequence of various vectors, such as vectors used for expression of recombinant proteins (“expression vectors”), vectors used for gene therapy, vectors used as vaccines, and the like. Such vectors may be, for example, plasmid vectors or viral vectors (i.e. vectors that comprise, or are derived from a viral genome). Methods for optimizing nucleotide sequences that encode recombinant proteins and which may be inserted into vector backbones, are described above. However, the methods of the present invention can also be used to optimize the vector backbone itself. For example, many vectors themselves encode various proteins. For example, viral vectors may encode various viral proteins. In some situations it may be desirable to optimize a vectors by eliminating or minimizing expression of proteins encoded by a vector backbone. In other situations it may be desirable to optimize a vector to increase expression of proteins encoded by a vector backbone. Vector sequences can be altered in the same ways as described above for protein-coding sequences in order to achieve these results. For example, the methods of the present invention may be used to identify sequence motifs present in the vector backbone that are either over- or under-represented as compared to the host genome. Preferably, the functional consequences of these sequence motifs should be determined. This can be done by mutating the sequence motifs in either the vector or in the host genome, and testing the effect of these mutations on certain biological properties such as rate of production of vector-encoded mRNAs, stability of vector-encoded mRNAs, rate or production of vector-encoded proteins, stability of vector-encoded proteins, and the like. Then, the nucleotide sequence of the vector backbone may be optimized by performing mutations to remove one or more disadvantageous sequence motifs in the vector backbone, or to add one or more advantageous sequence motifs to the vector backbone. Any suitable mutation methods known in the art, such as those described herein, may be used.

### Optimization of Vaccines

The methods described above for optimization of sequences for protein production and optimization of vector sequences can be used to optimize vaccines, including, but not limited to, attenuated viral vaccines, killed viral vaccines, viral vector vaccines, DNA vaccines, and protein vaccines.

Attenuated viruses are viruses that have been altered to weaken them, such that they no longer cause disease but may still stimulate an immune response. There are many ways in which a virus may be attenuated. For example, a virus can be attenuated by removal or disruption of viral sequences required for causing disease, while leaving intact those sequences encoding antigens recognized by the immune system. Attenuated viruses may or may not be capable of replication in host cells. Attenuated viruses that are capable of replication are useful because the virus is amplified in vivo after administration to the subject, thus increasing the amount of immunogen available to stimulate an immune response. The methods of the invention can be used to identify sequence motifs that are either under- or over-represented in a viral strain as compared to its host, and mutate these sequence motifs to increase the level of attenuation of a virus and/or to increase its immunogenicity in a host. For example, mutations can be made to disrupt or remove sequence motifs that are involved in the virulence of the viral strain or to add sequence motifs that suppress the virulence of the viral strain in its hosts. It is preferred that, if the attenuation methods used involve disrupting or deleting sequence motifs within the virus genome, these mutations are sufficiently large in size or number such that the chance reversion of the virus to a non-attenuated form is close to zero.

“Killed” or “inactivated” viral vaccines are generally non-functional and do not express viral genes or replicate in a vaccinated subject. However, the methods of the invention may be used to facilitate expansion and growth of a viral strain in vitro or ex vivo prior to inactivation of the virus. For example, by mutating one or more inhibitory sequence motifs in a virus, the rate of viral expansion in host cells may be increased, such that larger amounts of the virus can be produced in the host cells and then inactivated for use as a vaccine.

The methods of the present invention may also be used to optimize DNA vaccines and viral vector vaccines. For example, DNA vaccines or viral vector vaccines may comprise nucleotide sequences that encode certain immunogenic proteins in the context of a plasmid vector or viral vector backbone. The methods described above can be used to optimize expression of the nucleotide sequences that encode the immunogenic proteins, and also to optimize the sequence of the plasmid vector or viral vector backbone, for example by decreasing the expression of vector-encoded proteins.

The methods of the invention may also be used to optimize proteinaceous vaccines, such as proteinaceous vaccines produced by production of a recombinant proteins in a cellular host expression system. The methods described above can be used to optimized the nucleic acid encoding the protein for expression in the cellular host expression system.

### Mutation Methods

In some embodiments, the present invention involves mutating nucleotide sequences to add/create or remove/disrupt sequence motifs. Such mutations can me made using any suitable mutagenesis method known in the art, including, but not limited to, site-directed mutagenesis, oligonucletotide-directed mutagenesis, positive antibiotic selection methods, unique restriction site elimination (USE), deoxyuridine incorporation, phosphorothioate incorporation, and PCR-based mutagenesis methods. Details of such methods can be found in, for example, Lewis et al. (1990) Nucl. Acids Res. 18, p 3439; Bohnsack et al. (1996) Meth. Mol. Biol. 57, p 1; Vavra et al. (1996) Promega Notes 58, 30; Altered Sites®II in vitro Mutagenesis Systems Technical Manual #TM001, Promega Corporation; Deng et al. (1992) Anal. Biochem. 200, p 81; Kunkel et al. (1985) Proc. Natl. Acad. Sci. USA 82, p 488; Kunke et al. (1987) Meth. Enzymol. 154, p 367; Taylor et al. (1985) Nucl. Acids Res. 13, p 8764; Nakamaye et al. (1986) Nucl. Acids Res. 14, p 9679; Higuchi et al. (1988) Nucl. Acids Res. 16, p 7351; Shimada et al. (1996) Meth. Mol. Biol. 57, p 157; Ho et al. (1989) Gene 77, p 51; Horton et al. (1989) Gene 77, p 61; and Sarkar et al. (1990) BioTechniques 8, p 404. Numerous kits for performing site-directed mutagenesis are commercially available, such as the QuikChange® II Site-Directed Mutagenesis Kit from Stratgene Inc. and the Altered Sites® II in vitro mutagenesis system from Promega Inc. Such commercially available kits may also be used to mutate AGG motifs to non-AGG sequences

### Determination of Host-Pathogen Relationships

The methods and algorithms of the invention are well suited to studying the relationship between pathogens, such as viruses, and their hosts. For example, in the case of viruses, because the viral nucleic acid molecules are copied and expressed inside host cells, one might expect the viral and host genomes to be subject to some of the same evolutionary pressures. Thus, sequence motifs that are over-represented in a viral genome may also be over-represented in the genome of the viral host. Similarly, sequence motifs that are under-represented in a viral genome may also be under-represented in the genome of the viral host. Example 6 illustrates this phenomenon in bacteriophages and their host bacterial species, and shows that the genomes of bacteriophages scored highest with their correct bacterial host. Thus, the methods of the invention, in particular the scoring algorithms of the invention, can be used to score the genomes of pathogenic agents and score the genomes of potential host species, and identify the likely hosts of the pathogenic agents and/or identify the types of pathogenic agent likely to be able to infect a given host. For example, for a given pathogen, such as a virus, the scoring algorithm of the invention can be used to generate an overall score for a list of words L in a sequence from that pathogen, and compare that score to the scores for the same list of words in a scaled genome of various potential host species. Often pathogens will score highest with their natural hosts, and vice versa. In this way the likely hosts of pathogens can be determined, and conversely, the pathogens likely to infect a given host can be determined. Knowledge of these sequence motifs is also useful for several other applications. For example, drugs and vaccines can be designed to exploit these sequence motifs. These and other embodiments are described in more detail below.

Alternatively, in some circumstances, it is possible that sequence motifs that are over-represented in the genome of a pathogen may be under-represented in the genome of the pathogen's host, or conversely, that sequence motifs that are under-represented in the genome of a pathogen may be over-represented in the genome of the pathogen's host. This may occur, for example, if the pathogen gains a selective advantage from not containing the same sequence motifs as its host. For example, if the sequence motif is one that results in rapid degradation of mRNAs in the host species, a virus may be at a selective advantage if it does not contain this sequence motif, and can thus produce greater amounts of viral proteins. The Examples provided below describe the discovery of such a sequence motif in the genome of HIV using the methods and algorithms of the present invention. Knowledge of such sequence motifs is useful for several applications. For example, drugs and vaccines can be designed to exploit these sequence motifs. These and other embodiments are described in more detail below.

### Identification of Unique Phylogenetic Markers, and Determination of Phylogenetic Relationships

The present invention provides methods for identifying sequence motifs that are either over- or under-represented in a genome compared to that the frequency with which those motifs would be expected to occur by chance. The fact these sequences occur at frequencies other than would be expected in the absence of constraints, suggests that the motifs have been subject to selective pressure. For example, over-represented sequences are likely to have been selected for, and under-represented sequences are likely to have been selected against, during the evolution of the genome. Because of this, the sequence motifs identified using the methods of the invention can be used to classify organisms, viruses, or nucleotide sequences, or to determine the phylogenetic relationships between organisms, viruses, or nucleotide sequences. The scoring methods provided herein are also well suited for determining the phylogenetic relationships between organisms, viruses, or nucleotide sequences. Example 5 illustrates how the methods of the invention can be used to classify a genome and generate a phylogenetic tree.

### Other Applications

The algorithms and methods of the present invention have numerous other uses including, but not limited to, identification of splice sites, identification of exon splicing enhancers, identification of real exons, identification of mRNA degradation or stabilization signals, identification of transcription factor binding sites, and identification of sequences associated with tissue specificity.

The algorithms and methods of the invention could be used to identify sequences that are over- or underrepresented in real exons. For example, real exons are known to have overrepresented signals such as exon splicing enhancers. Such sequence motifs would be useful for helping to determine whether a given sequence is a real exon sequence or a confounding intronic sequence.

The algorithms and methods of the invention could also be used to identify mRNA stability or instability signals. The range of half-lives for different mRNAs spans two orders of magnitude, but the signals or structures that determine this difference in stability are unknown. The algorithms and methods of the invention could be used to identify these signals. For example, in one embodiment, the algorithms and methods of the invention could be applied to a first set of rapidly decaying mRNAs (for example the 1,000 most rapidly decaying mRNAs) and a second set of stable mRNAs (for example the 1,000 most stable mRNAs), and sequence motifs that are either over- or under-represented in the first set as compared to the second set could be identified. These sequence motifs could be mRNA stability or instability signals.

The algorithms and methods of the invention could also be used to identify tissue specificity signals. Evidence suggests that genes primarily expressed in certain tissues may have distinct properties, for example their codon usages and GC contents may be different. The methods of the present invention could be used to identify sequence motifs that are either over- or under-represented in genes that are expressed in a given tissue. Such signal motifs may also provide information about host tissue specificities and certain tissue tropic viruses.

These and other embodiments of the invention are further described in the following non-limiting examples. It should also be understood that numerous other variations of the embodiments described herein, including variations in the methods and algorithms described herein, are possible without departing from the spirit or scope of the invention. Such variations will be apparent to those of skill in the art

### EXAMPLES

### Example 1

### Algorithms for Identifying Sequence Motifs

Genome analysis has uncovered many sequence differences among organisms. Both mononucleotide and dinucleotide content, as well as codon usage, vary widely among genomes. The size of even small bacterial genomes is statistically sufficient to determine a substantially richer set of sequence-based features describing each organism. However, many of these features have remained elusive, in the coding regions in particular, due to complicated constraints. Each gene encodes a particular protein, which constrains its possible nucleotide sequence. Because the genetic code is degenerate, this constraint still allows for an enormous number of possible DNA sequences for each gene. Also, the overall codon usage in each gene is known to have strong biological consequences, possibly determined by isoaccepting tRNA abundances. In order to isolate new features within the coding regions, these constraints must be factored out.

To solve these problems, the present invention provides a “background genome” that shares the above-described constraints with a “real genome” but is otherwise random. The background genome encodes all the same proteins as the real genome, and the codon usage is precisely matched for each gene. Hidden sequence motifs in the real genome may be identified by identifying differences between the background genome and the real genome.

The present invention provides an algorithm that systematically computes the over- and underrepresented strings of nucleotides or “sequence motifs” in the real genome as compared to one or more background genomes. A major difficulty in finding these sequence motifs is that they are not independent. For example, if the motif ACGT is underrepresented, then ACGTA will also be underrepresented, as will ACG, etc. The assumption is that only one of these “words” has biological significance, while the other words are “along for the ride.” This problem extends to all words. As the set of words of a given length is finite and so are genomes, the frequency of any one word affects the frequency of all others. The present invention provides an iterative algorithm that uses an information theory measure to select the word contributing the most to the difference between the real and background genomes. At each step, the word is added to a list of over- or under-represented words and then its effects are factored out by resealing the background genome. In this way, a list of sequence motifs is obtained, each of which is likely to have biological significance, that contribute independently to the difference between the real and background genomes. The size of the genome affects the length of sequence motifs that can be resolved. For a typical bacterium such as Escherichia coli, sequence motifs of up to 7 nucleotides or more in length can be identified. In the methods of the invention, the amino acid order and codon usage of a gene are held fixed, so that the features uncovered by the algorithm are complementary to mononucleotide content and codon usage. For typical bacteria, the algorithm finds 100 to 200 sequence motifs of between 2 and 7 nucleotides in length (see Table 1). These previously unknown sequence motifs contain a wealth of biological information.

The following multistep method/algorithm was devised and used to identify sequence motifs that are under- or over-represented in a real genome. Flow charts illustrating the steps involved in these methods/algorithms are provided in FIG. 1 and FIG. 2.

### Step 1. Selection of a Real Genome

The first step was to select a real genome in which to identify sequence motifs. Data obtained using various different real genomes are presented in later Examples.

### Step 2. Generation of Background Genome

The next step was to generate a randomized background genome for comparison with the real genome. This was accomplished by randomly permuting the codons corresponding to each amino acid within every gene of the real genome, using the method described in Fuglsang, (2004) “The relationship between palindrome avoidance and intragenic codon usage variations: a Monte Carlo study” Biochem. Biophys. Res. Commun. 316: 755-762. A new coding sequence was created which had the same amino acid content and codon usage per gene as the real genome but was otherwise random.

### Step 3. Count the Occurrences of Each Word, w, in the Background Genome

The number of occurrences of each word, w, of length 2 to 7 nucleotides in the randomized background genome was counted. A length of 7 nucleotides was chosen as the maximum word length to consider based on the total length of the coding sequence of the bacterial genomes studied (see subsequent examples). However, other word lengths could have been used. Ideally, the average number of occurrences of each word should be much greater than zero in order for the algorithm to be robust, and so the maximum word length should be chosen such that, in the genome or genome portion being analyzed, words of that length will occur at a frequency much greater than zero.

In the specific examples described below, the procedure to generate random genomes and count the number of occurrences of each word was repeated 30 times, at which point the standard deviation in number of occurrences converged for the words. However, the procedure to generate random genomes could have been repeated more or less times.

### Step 4—Counts and Probabilities of Each Word in the Background Genome

The “average background count” N_{B}(w) of each word “w” across all 30 background genomes generated was calculated. The average background count for each word provides a measure of the number of occurrences of that word that would be expected to occur by chance in a real genome of the same size subject to the same constraints. We chose, for reasons that will be clarified below, to determine N_{B}(w) by considering only words of length 7 and generating the counts for smaller-length words by counting substrings.

The “average background count” N_{B}(w) was calculated as follows. We let L(w) equal the length of the word w, and we let C(W₇ⁱ, w) equal the number of times the string w is contained in the string W₇ⁱ of length 7. As an example, if w is AAC and W₇²⁵⁷ is AACAAAC, then L(w) equals 3 and C(W₇²⁵⁷, w) equals 2. The average background count for a given word of 7 nucleotides in length, N_{B}(W₇ⁱ), is equal to 1/30×(the sum of the number counts of that word, W₇ⁱ, in all 30 background genomes). The average background count for each word (includes words of lengths other than 7 nucleotides), N_{B}(w), was calculated according to equation (1) below.

$${N}_{B} ( w ) = {\sum }_{i = 0}^{( {4}^{7} - 1 )} \frac{{N}_{B} ( {W}_{7}^{i} ) \times C ( {W}_{7}^{i} , w )}{8 - L ( w )} \qquad ( 1 )$$

The counts for each word in the average background genome were then converted to frequencies (or equivalently probabilities), using the formula P_{B}(w)=N_{B}(w)/L, where L is the overall length of the coding sequence.

### Step 5—Counts and Probabilities of Each Word in the Real Genome

We also counted the number of occurrences of each word w in the real genome, to give N_{R}(w). The counts for each word in the real genome were then converted to frequencies (or equivalently probabilities), using the formula P_{R}(w)=N_{R}(w)/L, where L is the overall length of the coding sequence.

The two frequency distributions P_{B} and P_{R}, as calculated in steps 4 and 5 respectively, were used as the starting point in the word search algorithm described below. This word search algorithm generated a list of words contributing to the difference between the real genome and the background genomes, i.e. a list of sequence motifs that were either under or over-represented in the real genome as compared to the background genomes.

### Step 6. Iterative Word Search Algorithm

The word search algorithm used consisted of performing a first optional substep (A) to determine the distance between the real genome and background genome probability distributions, and then performing and repeating two additional substeps (B and C). In substep B the word that most significantly separated the real distribution from the background distribution was identified, based on a measure of significance S(w) described below. In substep C, the background probability distribution was rescaled to factor out the difference due to the word found in the first substep B. Substeps B and C were repeated a fixed number of times. However, alternatively, substeps B and C could have been repeated until the background distribution was sufficiently close to the real distribution.

### Substep A

The Kullback-Leibler distance D_{KL} between the real genome and background genome probability distributions was calculated using equation (2) below.

$${D}_{KL} = {\sum }_{i} {P}_{R} ( {W}_{7}^{i} ) log \frac{{P}_{R} ( {W}_{7}^{i} )}{{P}_{B} ( {W}_{7}^{i} )} \qquad ( 2 )$$

### Substep B

Next, the word that most significantly contributed to the distance/difference between the real genome distribution and the background genome distribution was identified, using a measure of significance, S(w), calculated using equation (3) below. S(w) measures the extent to which any one word w, of length 2 to 7, contributes to D_{KL}. Alternative methods of measuring the significance of any given word could also have been used.

$$S ( w ) = {P}_{R} ( w ) log \frac{{P}_{R} ( w )}{{P}_{B} ( w )} + [ 1 - {P}_{R} ( w ) ] log ( \frac{1 - {P}_{R} ( w )}{1 - {P}_{B} ( w )} ) \qquad ( 3 )$$

This can also be thought of as a Kullback-Leibler distance between two probability distributions, namely, the coarse-grained real and background distributions where we know only if a given word is or is not w. In the first step of the iteration, a word w of length 2 to 7 was chosen, which maximizes the significance measure S(w).

### Substep C

The next step was to rescale the background distribution in a minimal way such that the contribution of w became identical in both the real and background distributions, i.e. to factor out the contribution of w to the background genome. For the resealing to be minimal, the ratios of frequencies of words Wⁱ₇ of length 7 that contain w the same number of times should not change. That is, we wanted to rescale all words Wⁱ₇ with the same C(Wⁱ₇, w) by an equal factor. Therefore, it was necessary to work with an appropriate coarse graining of the detailed probability distributions. The distribution for the background was defined as the set of words Wⁱ₇ of length 7, with the probabilities P_{B}(Wⁱ₇). We partitioned this set of Wⁱ₇ into disjoint subsets where each element of a given subset contained the word w an equal number of times. These sets were as defined by equations (4) and (5) below.

K_{J}(w)={Wⁱ₇|C(Wⁱ₇,w)=J} (4)

with J={0, . . . , 6} and

K₀∪ . . . ∪K₆={W₇ⁱ} (5)

We wanted to rescale these disjoint subsets K_{J}(w) such that the probabilities of being in a given subset in the real and background distributions were equal.

$$\begin{matrix}{Q}_{R} ( {K}_{J} ) = {\sum }_{{W}_{7}^{i} ∈ {K}_{J}} {P}_{R} ( {W}_{7}^{i} ) & ( 6 ) \\ {Q}_{B} ( {K}_{J} ) = {\sum }_{{W}_{7}^{i} ∈ {K}_{J}} {P}_{B} ( {W}_{7}^{i} ) & ( 7 )\end{matrix}$$

These are well-defined probability distributions because they are grouped elements from the old probability distribution (and their probabilities are added). A resealing that factors out the contribution of w while conserving probability is given by

$${N}_{B} ( {W}_{7}^{i} ) \to \frac{{Q}_{R} ( {K}_{J} )}{{Q}_{B} ( {K}_{J} )} {N}_{B} ( {W}_{7}^{i} ) \qquad ( 8 )$$

where Wⁱ₇∪K_{J}, for all i. Note that with this rescaled distribution, the figure of merit for w is now S^{rescaled}(w)=0, because the contribution of w to the difference between the real and background genomes has been factored out. Stated another way, the contribution of w to D_{KL} has been removed.

Step 6A was then repeated to find the next word, w′, contributing most to the difference between the real and background genomes. Step 6B was then used to factor out the contribution of word w′, before repeating step Step 6A to find the next word, w″, and so on. Steps 6A and 6B were repeated iteratively to generate a list of words that contribute to the difference between the real and background genomes, i.e. to identify sequence motifs that are either under- or over-represented in the real genome as compared to the background genome.

With each successive round of this iterative algorithm, the background distribution converges to the real distribution. This is because D_{KL} is monotonically decreasing (see Example 2). D_{KL} is not negative, and is 0 if and only if the two distributions are identical. The algorithm of described in this step (step 6) can continue until convergence between the background distribution and real distribution is achieved, i.e. until the equation S(w)=0 for all w, which occurs when the real and background distributions are identical.

However, it is also possible to stop or cut off the algorithm at any desired stage, such as when the iterations are no longer contributing statistically significant words to the list. One possible cutoff could be the point where it becomes likely that chance fluctuations would create the most significant remaining word, appropriately corrected for multiple hypotheses [the set of all words of length L(w)]. Such a cutoff may occur when the selected word w satisfies equation (9) below, where (w) is the standard deviation of the background count for w.

$$erfc \{ \frac{[ {N}_{R} ( w ) - {N}_{B} ( w ) ]}{Δ ( w ) \sqrt{2}} \} \times {4}^{L ( w )} > 1 / 2 \qquad ( 9 )$$

However, in the present examples, the algorithms were stopped after 100 iterations, which is substantially below the cutoff calculated using equation 9.

### Example 2

### Proof that D_{KL} Decreases Monotonically with Rescaling

The following is a proof that D_{KL} decreases monotonically when background genomes are rescaled as described in step 6B of Example 1. Given two probability distributions {pj} and {qj}, with jεS and S being the set of possible outcomes, the Kullback-Leibler distance is given by equation (10) below.

$${D}_{KL} = {\sum }_{j} {p}_{j} log \frac{{p}_{j}}{{q}_{j}} \qquad ( 10 )$$

D_{KL} is non-negative and zero only if the distributions are identical.
Consider a disjoint partition of S, into r sets, S1 . . . Sr, as described by (11)

$${S}_{k} ⋂ {S}_{l} = Ø if k \neq l and {⋃}_{i} {S}_{i} = S \qquad ( 11 )$$

Next, define the coarse-grain probabilities,

$${P}_{i} = {\sum }_{j ∈ {s}_{i}} {p}_{j} and {Q}_{i} = {\sum }_{j ∈ {s}_{i}} {q}_{j} \qquad ( 12 )$$

Assume that Qi is >0 for all i. Note that both Pᵢ and Qᵢ are themselves probability distributions.
Define the rescaled distribution,

$${q}_{j} = {q}_{j} \frac{{P}_{i}}{{Q}_{i}} for J ∈ {S}_{i} \qquad ( 13 )$$

The new Kullback-Leibler distance is given by equation (14) below.

$$\begin{matrix}\begin{matrix}{D}_{KL}^{′} = {\sum }_{j} {p}_{j} log \frac{{p}_{j}}{{q}_{j}} \\ = {\sum }_{i} {\sum }_{j ∈ {s}_{i}} {p}_{j} log \frac{{p}_{j}}{{q}_{j} \frac{{P}_{i}}{{Q}_{i}}} \\ = {D}_{KL} - {\sum }_{i} {P}_{i} log \frac{{P}_{i}}{{Q}_{i}} \leq {D}_{KL}\end{matrix} & ( 14 )\end{matrix}$$

with equality only if Pᵢ equals Qᵢ for all i.

### Example 3

### Algorithms for Scoring Sequence Motifs

To score a coding sequence, S, of length s, with respect to a genome G of length g, a word list for G was first generated as described in Example 1, with the following modification: words were added to the list only if they would be significant for a sequence of length s. This significance was determined by resealing the counts and the standard deviations for each word to the scale s. The counts of each word in the background genome and the real genome were multiplied by s/g, which gives the expected counts, N_{b} and Nᵣ, for the sequence S. The standard deviation was rescaled by √s/g, giving Δ^{s}. If the word satisfied the equation |Nᵣ−N_{b}|>3×Δ^{s}, then it was included on the list; otherwise, it was skipped. Because s is much less than g, this standard was substantially more strict than the multiple-hypothesis corrected cut-off described in Example 1. The rest of the iterative procedure, including resealing the background distribution, was the same as that described in Example 1. This new list L formed the scoring template with the number of words X. To get the score, we formed the background B of the sequence S by the same Monte Carlo shuffling procedure used to generate the background genomes as described above. We then implemented the following iterative algorithm: at each step, we took a word W from the ordered list L. We then compared the counts of that word in the sequence S and the background B, adding 1 to our score only if the direction of the bias for W between S and B was the same as that for W between the genome G and its background, that is, only if W was over-represented in both G and S compared to their respective backgrounds, or was under-represented in both. We then rescaled B in the manner described above to factor out the effects of W, and proceeded to the next step. Going through the entire list L, we got a number Y out of X possible words for which there was agreement between the genome and the sequence. The final score was C×(X−Y/2)√Y, with C as a constant. For every short sequence, scoring was done for all 164 bacterial species in the NCBI database (http://www.ncbi.nlm.nih.gov/entrez/query.fcgi?db=Genome), which includes 253 chromosomes.

### Example 4

### Sequence Motifs Identified in Bacterial Genomes

The algorithm of Example 1 was used to identify a list of over and under-represented sequence motifs present in the genomes of all of the 164 bacterial species whose genomes are available in the NCBI databases, which includes 253 chromosomes. For most bacterial species, the algorithm identified between 100 and 200 words of between 2 to 7 nucleotides in length. Table 1 illustrates 100 of the over or under-represented sequence motifs identified in genome of the bacterium Escherichia coli (E. coli).

Table 1. E. coli Sequence Motifs

TABLE 1
E. coli word list

| | | Over- or |
|---|---|---|
| Sequence | | under- |
| no. | Sequence | represented |
| 1 | GGCC | − |
| 2 | TAG | − |
| 3 | GCTGG | + |
| 4 | TTGGA | − |
| 5 | CCC | − |
| 6 | GGCGCC | − |
| 7 | GTCC | − |
| 8 | GCCGGC | − |
| 9 | GGCG | + |
| 10 | GAGG | − |
| 11 | CCAAG | − |
| 12 | GGGT | − |
| 13 | TTGCC | + |
| 14 | CTAG | − |
| 15 | GCCAG | + |
| 16 | CTGCAG | − |
| 17 | AAGAG | + |
| 18 | ACTGG | + |
| 19 | GAAC | − |
| 20 | GTGT | − |
| 21 | TTGG | − |
| 22 | TCAAG | − |
| 23 | CGCTG | + |
| 24 | CGGTA | + |
| 25 | CACGTG | − |
| 26 | CCGCGG | − |
| 27 | GGCTC | − |
| 28 | CATG | − |
| 29 | CAGCTG | − |
| 30 | TCGGA | − |
| 31 | CGGCCG | − |
| 32 | AATT | − |
| 33 | TTGCT | + |
| 34 | CGAG | − |
| 35 | GGGGG | − |
| 36 | TCGTG | − |
| 37 | CTGAG | − |
| 38 | GACG | − |
| 39 | CTTG | − |
| 40 | GGTCTC | − |
| 41 | CCTGGA | − |
| 42 | CTCC | − |
| 43 | GCGGA | − |
| 44 | TCAGG | + |
| 45 | AGCGCT | − |
| 46 | TGGTG | + |
| 47 | TCGTA | − |
| 48 | CGCC | + |
| 49 | GGGCA | + |
| 50 | TTCCCG | + |
| 51 | CACCA | + |
| 52 | GGTACC | − |
| 53 | TTCG | − |
| 54 | TGGG | − |
| 55 | GAGACC | − |
| 56 | GATCG | − |
| 57 | CCCTG | − |
| 58 | CTGGCTG | + |
| 59 | CTGGCA | + |
| 60 | AAAAAAA | − |
| 61 | TGGCCT | + |
| 62 | GGAG | − |
| 63 | GAGCTC | − |
| 64 | CTCGAC | + |
| 65 | CAGCAA | + |
| 66 | AGAC | − |
| 67 | TCCAA | − |
| 68 | TATGAT | − |
| 69 | TATA | − |
| 70 | GGAC | − |
| 71 | TGGCCA | − |
| 72 | TTCCTCG | + |
| 73 | CTGTC | − |
| 74 | TGTG | − |
| 75 | TCTGA | − |
| 76 | CAGAT | − |
| 77 | CGTG | − |
| 78 | CAAG | − |
| 79 | CTGCTGG | − |
| 80 | TTTTTT | − |
| 81 | TCGCA | − |
| 82 | TATCG | + |
| 83 | TGCGA | − |
| 84 | CTGGGGC | + |
| 85 | TGAAG | + |
| 86 | GTCTGG | + |
| 87 | GTCGAT | + |
| 88 | GCATGC | − |
| 89 | GAAT | − |
| 90 | GTTGA | + |
| 91 | CAGTAA | + |
| 92 | GAGCC | − |
| 93 | ACGCT | + |
| 94 | CAGCGA | + |
| 95 | TCCGA | − |
| 96 | CTGGAAG | + |
| 97 | TCTTA | − |
| 98 | CTCAGA | − |
| 99 | TTGACA | − |
| 100 | CCATGG | − |

The lists of sequence motifs were generated from entire bacterial genomes. It was found that the sequence motifs identified were homogeneously distributed throughout the genomes as opposed to being clustered at particular locations. This was confirmed in two ways. First, using the bacterium E. coli as an example, we divided the genome in half and ran the algorithm on the two halves independently. The resulting lists of over- and under-represented words were the substantially the same for both halves of the genome, up to statistical fluctuations. For lists of 100 words, the top 80 words were found in both halves of the genome. This process was repeated multiple times with different divisions of the genome, and the results were similar.

As a second check that under- and over-representation of the words are not local features of the genome, an elementary algorithm the scoring algorithm described in Example 3 was used. This algorithm was created to score sequences of coding DNA based on the word lists from each genome. This algorithm takes as its inputs a coding DNA sequence and a list of words and assigns that sequence a score based on the under- and over-representation of the words in the sequence. The 253 bacterial chromosomes greater than 100 kb in length in the NCBI database were broken up into 50-kb and 100-kb portions. These sequences were scored separately against all 164 species. Ninety-two percent of the 100-kb slices scored highest with their own species. Using 50-kb sequences, 86% scored best with their own species. This confirms that the words correspond to features that are homogeneous throughout each bacterial genome. Neither GC content nor codon usage has this property of homogeneity; both vary substantially within single genomes.

### Example 5

### Classification of Bacterial Sequences and Phylogenetic Relationships

As described above in Example 5, when the 253 bacterial chromosomes greater than 100 kb in length in the NCBI database were broken up into 50-kb and 100-kb portions, and scored separately against all 164 species, 92% of the 100-kb portions and 86% of the 50-kb portions scored highest with their own species. This outcome suggests that the sequence motifs identified using the methods of the present invention are useful classifiers of sequences. For instance, the sequences attained from microbes in the Sargasso Sea described by Venter et al. (9) can be compared with known bacteria without requiring homologous genes. Prior to the present invention, the best-known bacterial genome classifier was an oligonucleotide approach developed by Karlin and Cardon [6]. Using the scoring algorithm of the present invention, the classification results for 50 kb and 100 kb genome portions were slightly better than those obtained with the most-comprehensive oligonucleotide approach, which involves comparing frequencies of oligonucleotides with lengths up to 4. The scoring system of the present invention was also substantially better at classifying sequences than the dinucleotide approach applied by Venter et al. [9]

The scoring algorithm of the present invention was also adapted to measure distance between genomes. The metric utilized 50-kb portions of genomes and the scoring method described in the above Examples. The distance between two genomes, A and B, was calculated in three steps. First, all of the 50-kb portions of genome A were scored against the full genome B, and then the scores were averaged. The same process was repeated for the 50-kb portions of genome B, scored against genome A. Next, the two averages were symmetrized. Lastly, the symmetrized score was subtracted from the maximum possible score. This distance has most of the properties of a metric-symmetric, positive definite zero only if A equals B, although it does not obey the triangle inequality. We used nearest-neighbor clustering and employed the “PHYLIP” software package to generate the phylogenetic trees provided herein (3). “PHYLIP” or the “PHYLogeny Inference Package” is a package of programs for inferring evolutionary trees. It is available free on the internet at http://evolution.genetics.washington.edu/phylip.html.

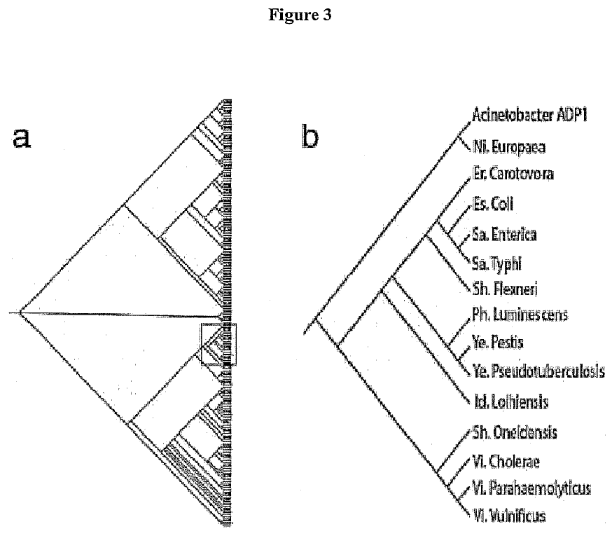

Applying hierarchal clustering to a matrix of the distances between the set of 164 bacterial species, calculated as described above, a phylogenetic tree was generated (FIG. 3). This tree captured most of the standard bacterial taxonomy. For example, FIG. 3 part (b) shows that the majority of Enterobacteria are grouped correctly in the same lade using the methods of the invention. This suggests that the properties encoded by the sequence motifs are conserved evolutionarily. Since the distance measure used herein is based on a whole-genome property, some of the common pitfalls associated with making phylogenetic trees, like lateral gene transfer, were avoided. Also, this method allowed the addition of new species in the tree without requiring any homologous genes or even a large amount of sequenced genomes.

### Example 6

### Determination of Virus-Host Relationships

The methods and algorithms of the invention are also well suited to studying the relationship between viruses and their hosts. Since virus DNA (or RNA) is copied and expressed inside a host, one might expect that viruses and their hosts share some evolutionary pressures. However, mononucleotide contents and codon usages differ dramatically between hosts and bacteriophages. Some information has been gained from oligonucleotide comparisons, but the scoring system described in the algorithms described in the above example are more than 60% better. Out of the set of sequenced DNA bacteriophage (or “phage”) genomes available on the NCBI website, 185 of the phages have known primary hosts. Many of the phages are known or suspected to have multiple host species within the same genus. For this reason, host genomes were considered at the genus level. The 164 species of bacterial hosts divide into 108 different genera. Using the algorithms described in the above example, the correct host genus scored highest for 93 out of the 185 phages, and 131 phages had the correct host in the top three scores (see Table 2).

TABLE 2
Scoring of phage host predictions

% of phages with:

| | | Top three |
|---|---|---|
| Phage type | Top score | scores |
| All | 50 | 71 |
| dsDNA | 58 | 82 |
| Lytic | 45 | 59 |
| Temperate dsDNA | 70 | 93 |

For comparison, the best oligonucleotide scoring system identifies only 58 of 185 host genera correctly. Furthermore, both codon usage and mononucleotide content are poor predictors of phage hosts.

By restricting the analysis to double-stranded DNA (dsDNA) phages, which comprise the large majority of known phages, the host predictions were improved further. Removing the 35 single-stranded DNA phages improved the scoring to 87/150 or 58% for the top score and 123/150 or 82% for the top three scores. The phages can be further classified as either temperate or lytic phages using the methods of the invention. For temperate dsDNA phages, which constitute the majority of sequenced phages, the prediction of hosts achieved using the methods of the present invention was excellent (93% in the top three, with 70% with the top score). For lytic phages, the results were not as good, although still better than 50% in the top three, suggesting that their DNA is not subject to the same evolutionary pressures as those of the host cell.

### Example 7

### Identification of a Sequence Motif in Lentiviral Genomes

Lentiviruses belong to the retrovirus family of viruses. The term “lenti” is Latin for “slow”. Lentiviruses are characterized by having a long incubation period and the ability to infect neighboring cells directly without having to form extracellular particles. Their slow turnover, coupled with their ability to remain intracellular for long periods of time, make lentiviruses particularly adept at evading the immune response in infected hosts. It has been suggested that these properties of lentiviruses may be due, at least in part, to the presence of one or more inhibitory nucleotide signal sequences or “INS” sequences, in lentiviral genomes.

The algorithm described in Example 1 was used to look for sequence motifs that are over- or under-represented in the HIV-1 genome as compared to genes in the human genome that have a comparable A-rich content (the HIV genome has a high A-content). 4,000 human genes having A-contents comparable to HIV were identified and studied using the algorithms described above. A trinucleotide sequence motif (AGG) was identified that was under-represented in these human genes as compared to the expected frequency. The same AGG sequence motif was found to be over-represented in both the HIV-1 genome, Of 48 AGG oligonucleotide sequences identified in the HIV-1 gag gene, over two thirds were not in the reading frame that encodes an amino acids, suggesting that these sequences were not conserved due to selective pressure at the amino acid/protein level. The AGG motif was also found to be particularly conserved even in the third position of codons. Furthermore, the AGG motif was also found to be over-represented in over 400 different HIV-1 strains analyzed, and in the genomes of other lentiviruses including HIV-2, several strains of simian immunodeficiency virus (SIV), feline immunodeficiency virus (FIV) and equine infectious anemia virus (EIAV). These results suggest that the AGG motif may have been selected against in the human genome (i.e. in the HIV host), while being retained and/or enriched in lentiviral genomes. The AGG motif may be an INS sequence. This can be tested by mutating one or more the AGG sequence motifs in a lentiviral genome and observing the effects on the biology of the virus.

### Example 8

### Vaccines

To date, there is no commercially available vaccine capable of conferring immunity against HIV challenge. There are many reasons why it has not been possible to generate such a vaccine. One factor that may have contributed to the difficulty in producing a vaccine could be the ability of HIV to remain intracelleular for extended periods of time. Intracellular virus is protected from antibody-mediated (but not CD-8 T-cell-mediated) immunity. The HIV virus is able to remain hidden intracellularly for long periods because of its slow rate of production inside cells, its ability to remain latent inside cells, and its ability to spread from cell to cell by cell fusion provides.

These properties of the HIV virus may adversely affect the ability to generate an effective vaccine on multiple levels. On one level, vaccines based on the HIV virus, such as inactivated or attenuated HIV vaccines, may enter and remain in host cells for extended periods of time, as do wild type HIV viruses. Thus, because of the slow life cycle of the virus and the limited amount of time during which the viruses are exposed extracellularly, the immune system is not able to generate an immune response strong enough to provide protective immunity against subsequent challenge with HIV. On another level, DNA may express very low levels of HIV-encoded antigens due to the presence of INS sequences, such as AGG motifs, in the nucleic acid constructs used. Generally, the more antigen that is produced, the greater the immune response will be. Thus, if low levels of HIV-antigens are produced, the immune response generated against those antigens will also be low.

It may be possible to overcome these problems, and thus generate more effective vaccines, by mutating one or more AGG motifs within the lentiviral nucleic acids used in, or used to produce, vaccines. For example, an attenuated HIV vaccine could be produced which in addition to being altered so as to reduce its ability to cause disease, is also mutated to disrupt on or more AGG motifs.

To test the above approaches, attenuated HIV viruses having mutated AGG motifs will be generated. The ability of these mutated viruses to infect host cells, express the encoded HIV proteins, and produce new virus particles will be studied in vitro using cell culture systems. Also, the ability of these mutated viruses to generate an immune response in a host in vivo, will be tested using suitable animal models of HIV infection.

In addition, the same approach will be tested using the SIV virus and the FIV virus. Attenuated FIV and SIV viruses having mutated AGG motifs will be generated. The ability of these mutated viruses to infect host cells will be studied in vitro using cell culture systems. Also, the ability of theses mutated viruses to generate an immune response will be tested in vivo in hosts susceptible to SIV and/or HIV infection. These SIV and FIV experiments will provide useful models for HIV vaccines/HIV infection. Additionally, the generation and testing of vaccines against SIV in simian species, and the generation and testing of vaccines against FIV in feline species, are useful in of themselves.

### Example 9

### Sequence Motif Binding Proteins and Agents

The sequence motifs of the present invention may be binding sites for proteins. Having identified a sequence motif using the methods and algorithms of the invention, it will be possible identify and isolate such proteins. For example, cell or tissue extracts can be passed over columns that contain the sequence motifs of the invention, with washes of non-specific and/or competitor DNA if necessary. If the cell or tissue extracts contain a protein that binds specifically to the sequence motif, this protein will be retained on the column, and can subsequently be eluted from the column and purified. This would also enable the amino acid sequence of the protein to be determined, and the gene encoding the protein to be identified.

If identified, proteins that bind to the sequence motifs of the invention, or agents that mimic the effects of theses proteins by binding to the sequence motif, could be useful for various applications.

### Example 10

### Other Potential Applications of the Methods and Algorithms of the Invention

Some possible uses for the methods and algorithms of the invention include identification of splice sites, exon splicing enhancers, mRNA degradation or stabilization signals, transcription factor binding sites, and sequences associated with tissue specificity. For example, real exons have overrepresented signals, such as exon splicing enhancers. The algorithms and methods of the invention could be used to determine a comprehensive list of over- and underrepresented sequences in real exons, which could be used to separate real exons from confounding intronic sequences. For mRNA stability, a few groups have measured the decay rates for large numbers of mRNAs in a variety of organisms, including humans. The range of mRNA half-lives spans two orders of magnitude, but the signals or structures that determine this difference in stability are unknown. If the algorithms and methods of the invention are applied to a set of, for example, the 1,000 most rapidly decaying mRNAs and, for example, the 1,000 most stable mRNAs, the differences in the two lists should provide a set of important signals. For tissue specificity, it has been shown in the last couple of years that genes primarily expressed in different tissues have distinct properties; their codon usages and GC contents are different. The methods and algorithms of the invention could be used to find additional signals that distinguish tissues. These signals also have the potential to provide information about the host tissue specificities and preferences for particular viruses. Unlike codon usage and mononucleotide content, which are not shared by phages and their bacterial hosts (or by human viruses and their host tissues), the methods and algorithms of the present invention are excellent predictors of viral hosts.

The methods and algorithms of the present invention can also be used to help find transcription factor binding sites. From the DPInteract database (http://arep.med.harvard.edu/dpinteract/), we extracted the set of known binding sites for the 13 transcription factors that had 15 or more binding sites listed for E. coli. The binding sites determined a set of weight matrices that score the binding motifs. By running the weight matrices over the real E. coli genome and comparing them with the background E. coli genome, we found that 12 of the 13 motifs were significantly (4 standard deviations) underrepresented in the coding region. This procedure can be used as a filter to decide whether a motif is real, which is of immediate utility, as the commonly used motif finders pick out excess signals that are not real transcription factor binding motifs.

The background genomes of the present invention may also be useful in their own right. Many bioinformatics problems require searching for a longer motif or sequence by comparing it with a random background. These problems have proved difficult because there is no procedure to generate a background model that includes all of the biases in real genomes. The algorithm and background genomes of the present invention determine and take into account all of the short global biases. Creating a background model that respects these biases will allow a variety of difficult bioinformatics problems to become tractable.

1-80. (canceled)

81. A method for identifying one or more sequence motifs that are over-represented or under-represented in a real genome or real genome portion as compared to the frequency of those sequence motifs that would be expected to occur by chance, the method comprising:
(i) selecting a real genome or real genome portion in which to identify over-represented or under-represented sequence motifs,
(ii) generating a background genome that encodes the same amino acids, and has the same codon usage as the real genome or real genome portion, but is otherwise random,
(iii) identifying, and counting the number of occurrences, of each word of a given length in the background genome,
(iv) counting the number of occurrences of each word identified in part (iii) in the real genome or real genome portion,
(v) performing an algorithm to identify words contributing to the difference between the background genome and the real genome or real genome portion, wherein the algorithm comprises the following the steps:
(a) identifying the word most significantly contributing to the difference between the real genome or real genome portion and the background genome,
(b) resealing the background genome to factor out the difference between the real genome or real genome portion and the background genome that was due to the word identified in step (a), and
(c) optionally repeating steps (a) and (b) to identify additional words contributing to the difference between the real genome or real genome portion and the background genome,
wherein the words identified in each repetition of step (a) are sequence motifs that are over-represented or under-represented in the real genome or real genome portion as compared to the frequency of those sequences that would be expected to occur by chance.

82. The method of claim 81, wherein the words are from two to ten nucleotides in length.

83. The method of claim 81, wherein step (ii) is performed using a Monte Carlo algorithm.

84. The method of claim 81, wherein steps (ii) and (iii) are repeated until the standard deviation for the number of occurrences of the words converges.

85. The method of claim 81, wherein step (v)(a) comprises:
(i) calculating the Kullback-Leibler distance, D_{KL}, between the real genome and background genome, and
(ii) identifying the word that most significantly contributes to D_{KL}.

86. The method of claim 81, wherein steps (v)(a) and (v)(b) are repeated until the real genome and background genome converge.

87. The method of claim 81, wherein steps (v)(a) and (v)(b) are repeated until the Kullback-Leibler distance, D_{KL}, between the real genome and background genome reaches zero.

88. The method of claim 81, wherein steps (v)(a) and (v)(b) are repeated X times to identify X sequence motifs, wherein X is a whole number between 1 and 100.

89. A method for optimizing the production of a protein in a host, the method comprising:
(a) identifying one or more sequence motifs that are either under-represented or over-represented in a host genome or genome portion, as compared to the frequency of those sequences that would be expected to occur by chance,
(b) obtaining a nucleotide sequence encoding a protein to be expressed in the host,
(c) mutating the nucleotide sequence encoding the protein to reduce the number of those sequence motifs that are under-represented in the host genome or genome portion, or to increase the number of those sequence motifs that are over-represented in the host genome or genome portion, or both,
wherein the mutations result in improved production of the protein in the host.

90. The method of claim 89, wherein the amino acid sequence encoded by the nucleotide sequence does not change following mutations made in step (c).

91. The method of claim 89, wherein the protein is a therapeutic protein.

92. The method of claim 89, wherein the protein is an immunogenic protein.

93. The method of claim 92, wherein the protein is suitable for use in a vaccine composition.

94. The method of claim 89, wherein the step of identifying one or more sequence motifs that are either under-represented or over-represented in a host genome or genome portion, as compared to the frequency of those sequences that would be expected to occur by chance, comprises:
(i) obtaining the nucleotide sequence of the host genome,
(ii) generating a background genome that encodes the same amino acids, and has the same codon usage as the host genome, but is otherwise random,
(iii) identifying, and counting the number of occurrences of each word of a given length in the background genome,
(iv) counting the number of occurrences of, each word identified in part (iii) in the host genome,
(v) identifying the word most significantly contributing to the difference between the host genome and the background genome,
(vi) resealing the background genome to factor out the difference between the host genome and the background genome that was due to the word identified in step (v), and
(vii) optionally repeating steps (v) and (vi) to identify additional words contributing to the difference between the host genome and the background genome,
wherein the words identified in each repetition of step (v) are sequence motifs that are over-represented or under-represented in the real genome as compared to the frequency of those sequences that would be expected to occur by chance.

95. A method for comparing a first sequence, S1, to second sequence, S2, the method comprising:
(a) identifying one or more words that are either under-represented or over-represented in a first sequence, S1, as compared to the frequency of those words that would be expected to occur by chance,
(b) determining whether any of the words identified in step (a) are either under-represented or over-represented in a second sequence, S2, as compared to the frequency of those words that would be expected to occur by chance,
(c) generating a score calculating for the similarity between S1 and S2 based on the number of words, out of the total number of words identified in step (a), that are either over-represented in both S1 and S2, or are under-represented in both S1 and S2,
wherein the higher the score the greater the similarity between sequence S1 and sequence S2.

96. The method of claim 95, wherein the words are identified using the methods of claim 1.

97. The method of claim 95, wherein S1 and S2 are sequences from two different organisms or viruses, and wherein the higher the score the closer the phylogenetic relationship between S1 and S2, and the lower the score, the more distant the phylogenetic relationship between S1 and S2.

98. The method of claim 95, wherein S1 is a sequence from a host, and S2 is a sequence from a pathogenic agent, and wherein the higher the score, the more likely it is that the host organism is susceptible to infection by the pathogenic agent.

99. The method of claim 95, wherein S1 is a sequence from a host, and S2 is a sequence S from a pathogenic agent, and wherein the higher the score, the more likely it is that the pathogenic agent is capable of infecting the host.

100. A method for comparing a first sequence S1 of length s1, to a second sequence S2 of length s2, the method comprising:
(a) generating a list of words that are either under-represented of over-represented in a sequence S1 of length s1, as compared to the frequency of those words that occur in a background genome, B_{S1}, that encodes the same amino acids, and has the same codon usage as S1, but is otherwise random,
(b) generating a list L of words W, wherein each of the words W is a word identified in step (a), whose under- or over-representation would be statistically significant in a coding sequence of length s2,
(c) generating a background sequence B_{S2} that encodes the same amino acids, and has the same codon usage as the sequence S2, but is otherwise random,
(d) performing an iterative algorithm comprising the following steps:
(i) taking a word W from the list L,
(ii) adding a numerical score of “one” for that word only if the word is over-represented in both S1 and S2 compared to their respective backgrounds B_{S1}, and B_{S2}, or if the word is under-represented in both S1 and S2 compared to their respective backgrounds B_{S1} and B_{S2},
(iii) resealing the background B_{S2} to factor out the effects of W, and
(iv) repeating steps (i) to (iii) for each word Win the list L, to produce a list of Y words having a score of one or more out of X possible words in the list W,
(e) calculating a final score based on the number of sequence motifs having a score of one or more out of the total number of sequence motifs identified in step (a),
wherein the higher the final score the greater the similarity between sequence S1 and sequence S2.
